(19) United States
(12) Patent Application Publication
(10) Pub. No.: US 2026/0013454 A1
(43) Pub. Date: Jan. 15, 2026

(54) QUANTITATIVE TRAIT LOCUS ASSOCIATED WITH A PATHOGEN RESISTANCE TRAIT IN CANNABIS

(71) Applicant: Puregene AG, Zeiningen (CH)

(72) Inventors: Claudio Cropano, Zeiningen (CH); Dániel Árpád Carrera, Zeiningen (CH); Gavin Mager George, Zeiningen (CH); Leron Katsir, Zeiningen (CH); Marc Schmid, Zeiningen (CH); Maximilian Moritz Vogt, Zeiningen (CH); Michael Eduard Ruckle, Zeiningen (CH); Michele Wyler, Zeiningen (CH)

(21) Appl. No.: 18/994,531

(22) PCT Filed: Aug. 11, 2023
(86) PCT No.: PCT/IB2023/058132

§ 371 Date: Jan. 14, 2025

(30) Foreign Application Priority Data
Aug. 11, 2022 (GB) 2211773.3

(51) Int. Cl. A01H 1/04; A01H 1/00; C12Q 1/686; C12Q 1/6869; C12Q 1/6895

(52) U.S. Cl. CPC A01H 1/045; A01H 1/1255; C12Q 1/686; C12Q 1/6869; C12Q 1/6895; C12Q 2600/13; C12Q 2600/156

(57) ABSTRACT

The invention relates to methods of identifying and characterizing a Cannabis spp. plant comprising a quantitative trait locus (QTL) or a causative polymorphism associated with pathogen resistance, and to Cannabis spp. plants having a pathogen resistance trait of interest comprising defined allelic states of polymorphisms defining the QTL. Also provided are plants with a pathogen resistance trait of interest identified by the methods described herein. The invention further relates to marker assisted selection and marker assisted breeding methods for obtaining plants having a pathogen resistance trait of interest, as well as to methods of producing Cannabis spp. plants with the pathogen resistance trait of interest and plants produced by these methods, based on the allelic state of the QTLs or the causative polymorphism.

### BACKGROUND OF THE INVENTION

The invention relates to methods of identifying and characterizing a Cannabis spp. plant comprising a quantitative trait locus (QTL) associated with pathogen resistance, including to Botrytis cinerea, and to Cannabis spp. plants having a resistance trait of interest comprising defined allelic states of the polymorphisms defining the QTL. The invention further relates to plants with a pathogen resistance trait of interest identified by the methods described herein. The invention also relates to marker assisted selection and marker assisted breeding methods for obtaining plants having a pathogen resistance trait of interest. Also provided are methods of producing Cannabis spp. plants with the pathogen resistance trait of interest and plants produced by these methods, based on the allelic state of the QTLs, as well as genes responsible for controlling the pathogen resistance trait of interest.

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are typically used for different purposes. Hemp is primarily used for industrial purposes, for example in feed, food, seed, fiber, and oil production. Alternatively, HRT cannabis is largely cultivated and bred for high concentrations of the pharmacological constituents, cannabinoids, produced largely in the trichomes. Biomass, primarily the leaf, of cannabis can also be an important source of cannabinoids.

Cannabis is one of a few species in the plant kingdom to produce phytocannabinoids. Phytocannabinoids are a class of terpenoid acting as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

HRT cannabis is predominantly cultivated in controlled indoor or greenhouse environments while hemp is majorly cultivated in field, both cannabis types are grown globally in varied geographical settings. These varied environments expose cannabis to a wide range of potential pathogens that can impact negatively on harvest and yield. Pathogens that impact flower quality in field settings is a major driver for HRT cannabis being grown in controlled environments. Because of prohibition breeding HRT and Hemp type cannabis for resistance to potential pathogens has been limited. Particularly for HRT cannabis, altering the growth environment has been generally used in place of breeding for resistance. With the lifting of prohibition in many regions, cultivation is occurring in new environments leading to exposure to a range of new pathogen challenges. It is estimated that more than 90 different fungal species can cause disease in cannabis.

The fungal pathogen Botrytis cinerea (Botrytis) referred to as grey mould is of the most significant hindrances to cultivation of cannabis in controlled environment and in field cannabis production systems. Botrytis is an air-borne necrotrophic fungus that produces spreading lesions covered by a grey mat of conidia on leaves, flowers, and stems that can lead to rapid decay of cannabis plants. Flowers of cannabis in their later stages of development and at post-harvest are particularly susceptible.

Botrytis secretes lytic enzymes in order to compromise and invade plant tissue, this is followed by the synthesis of phytotoxic metabolites to trigger an oxidative burst and the induction of host programmed cell death. Infected plant cells collapse allowing nutrients to be extracted for fungal growth. Environmental factors like humidity can greatly impact success pathogen success, outdoor hemp yield can be reduced by Botrytis up to 32% in rainy years. Botrytis, and other pathogens, can continue to propagate on harvested cannabis flowers contributing to significant post-harvest losses. While the economic impact of Botrytis on Cannabis has not been globally evaluated, in comparison 14-45% of ornamental flowers are lost due to Botrytis in post-harvest storage. Furthermore, Botrytis is recognized globally as being one of the most significant plant pathogens—causing billions of dollars of losses related to reduced harvest yield and loss post-harvest.

Management of Botrytis is often approached by identifying resistant varieties. Many varieties are still susceptible and high fungicidal treatments are employed in addition to creating environmental conditions where Botrytis does not thrive. Fungicidal treatment is often not an option for producers concerned about consumer preference and health. Plant management can significantly reduce loss but can be cost intensive. Extending resistance to all susceptible varieties could offer producers of both flower-type and hemp type cannabis solutions to reducing the impact of Botrytis, improving flower quality and yield, without relying on fungicidal treatment. However, breeding for resistant varieties in hemp has been slow to advance and in HRT cannabis is not pursued.

The molecular basis for fungal disease resistance in Cannabis is fundamentally unknown, hindering breeding by marker based selection and population based pre-screening. Furthermore HRT- and hemp cannabis genotypes that are resistant to Botrytis have not been described. In model species like Arabidopsis thaliana research identified the phytohormones jasmonic acid, abscisic acid, salicylic acid, and ethylene as key players in this defense response. The cross talk between these hormones is often altered in mutant plants that are more resistant or susceptible. This underlies that the defense response to fungal pathogens is complex, polygenic and involves overlapping and redundant signaling pathways. Several genes linked to jasmonate and ethylene hormone signaling, have been suggested as candidate genes for improving disease resistance in Arabidopsis, however no equivalent candidate genes have been identified in Cannabis.

Because there are no good methods for selecting disease resistant cannabis seedlings based on morphological indicators early on, molecular markers would be useful for assessing disease resistance and susceptibility at the seedling stage in order to use those selections for production or as the basis for breeding.

Selection of cannabis plants with resistance to disease caused by these pathogens is challenging for breeders and producers as plants may not exhibit symptoms of the disease until later stages of growth, after flower, or sometimes not at all because the pathogen is not present. As such, the identification of molecular markers to identify and select for and against cannabis plants that may have increased disease resistance would be a significant contribution to the cannabis industry by lowering the prevalence and damage associated with fungal pathogens in HRT and fiber-type cannabis production systems.

Traditional resistance breeding strategies, while potentially useful, are slower and can result in the loss of favorable linked characteristics.

In the present invention, several genetic regions in cannabis that significantly associate with cannabis resistance to B. cinerea were identified from a parent population displaying such resistance, with the aim of developing varieties with a stabilized B. cinerea resistance phenotype. The markers were also shown to be useful in breeding cannabis spp. plants displaying resistance to other pathogens.

### SUMMARY OF THE INVENTION

The present invention describes methods of identifying or characterizing a Cannabis spp. plant comprising a quantitative trait locus (QTL) associated with resistance to biotic pathogens, and to Cannabis spp. plants comprising the QTL of interest. The invention also relates to disease resistant plants identified by the methods. The invention further relates to marker assisted selection and marker assisted breeding methods for obtaining plants that have general disease resistance, as well as to methods of producing disease resistant Cannabis spp. plants and to the disease resistant plants produced by these methods.

According to a first aspect of the present invention there is provided for a method for characterizing a Cannabis spp. plant with respect to a pathogen resistance trait, and in particular to a Botrytis resistance trait, the method comprising the steps of: (i) genotyping at least one plant with respect to at least one pathogen resistance QTL by detecting one or more polymorphisms associated with the pathogen resistance trait as defined in Table 2 or 9; and (ii) characterizing the one or more plants with respect to the at least one pathogen resistance QTL as having an increased pathogen resistance QTL, a decreased pathogen resistance QTL or an intermediate pathogen resistance QTL based on the genotype at the polymorphism.

In a first embodiment of the method for characterizing a Cannabis spp. plant with respect to a pathogen resistance phenotype, the polymorphism may be selected from the group consisting of “common_3407”, “common_703”, “common_1691”, “common_701”, or combinations thereof as defined in Table 2 or 9. These markers have all been shown to have particularly high predictive value for the pathogen resistance QTL and trait.

In a second embodiment of the method for characterizing a Cannabis spp. plant with respect to a pathogen resistance phenotype, the genotyping may be performed by any PCR-based detection method using molecular markers, by sequencing of PCR products containing the one or more polymorphisms, by targeted resequencing, by whole genome sequencing, or by restriction-based methods, for detecting the one or more polymorphisms.

In a third embodiment of the method for characterizing a Cannabis spp. plant with respect to a pathogen resistance phenotype, the molecular markers may be for detecting polymorphisms at regular intervals within the at least one pathogen resistance QTL such that recombination can be excluded. In an alternative embodiment, the molecular markers may be for detecting polymorphisms at regular intervals within the at least one pathogen resistance QTL such that recombination can be quantified to estimate linkage disequilibrium between a particular polymorphism and the pathogen resistance phenotype. It will be appreciated by those of skill in the art that several possible markers may be designed for detecting the polymorphisms. For example, molecular markers may be for detecting polymorphisms such that recombination events can be detected to a resolution of 10′000 or 100′000 or 500′000 base pairs within the QTL. In one embodiment, the molecular markers may be designed based on a context sequence for the polymorphism described in Table 3 or 10, or may be selected from the primer pairs as defined in Table 4 or 11. In another embodiment, the molecular markers may further be designed based on a context sequence for a causative polymorphism described in Table 8 or a genetic marker linked to the QTL described in Table 13.

In a fourth embodiment of the method for characterizing a Cannabis spp. plant with respect to a pathogen resistance phenotype, the pathogen resistance QTL may be selected from the group consisting of:
i. a quantitative trait locus having a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL, such as a genetic marker described in Table 13;
ii. a quantitative trait locus having a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL;
iii. a quantitative trait locus defined by a single nucleotide polymorphism at position 79997 of NC_044373.1 of the CS10 genome and associated with pathogen resistance as defined in Table 2, or a genetic marker linked to the QTL; and/or
iv. a combination of any of (i) to (iii).

In a fifth embodiment of the method for characterizing a Cannabis spp. plant with respect to a pathogen resistance trait, the pathogen resistance trait is a Botrytis cinerea resistance trait, and the pathogen resistance QTL is a Botrytis resistance QTL.

According to a second aspect of the present invention, there is provided for a method of producing a Cannabis spp. Plant having a pathogen resistance trait of interest, the method comprising the steps of: (i) providing a donor parent plant having in its genome at least one pathogen resistance QTL characterized by one or more polymorphisms associated with the pathogen resistance trait of interest as defined Table 2 or 9; (ii) crossing the donor parent plant having the at least one pathogen resistance QTL with at least one recipient parent plant to obtain a progeny population of cannabis plants; (iii) screening the progeny population of cannabis plants for the presence of the at least one pathogen resistance QTL; and (iv) selecting one or more progeny plants having the at least one QTL, wherein the mature plant displays the pathogen resistance trait of interest. The pathogen resistance trait of interest may be an increased pathogen resistance trait, a decreased pathogen resistance trait, or an intermediate pathogen resistance. In this way, the trait can be modulated in a plant using the pathogen resistance QTL of the invention.

In a first embodiment of the method of producing a Cannabis spp. Plant having a pathogen resistance trait of interest, the method may further comprise the steps of: (v) crossing the one or more progeny plants with the donor recipient plant; or (vi) selfing the one or more progeny plants.

According to a second embodiment of the method of producing a Cannabis spp. Plant having a pathogen resistance trait of interest, the screening may comprise genotyping at least one plant from the progeny population with respect to the at least one pathogen resistance QTL by detecting one or more polymorphisms associated with the pathogen resistance trait of interest as defined Table 2 or 9.

In a third embodiment of the method of producing a Cannabis spp. Plant having a pathogen resistance trait of interest, the method my comprise a step of genotyping the donor parent plant with respect to the at least one pathogen resistance QTL by detecting one or more polymorphisms associated with the pathogen resistance trait of interest as defined Table 2 or 9, prior to step (i).

According to a fourth embodiment of the method of producing a Cannabis spp. Plant having a pathogen resistance trait of interest, the genotyping may be performed by a PCR-based detection using molecular markers, by sequencing of PCR products containing the one or more polymorphisms, by targeted resequencing, by whole genome sequencing, or by restriction-based methods, for detecting the one or more polymorphisms.

In a fifth embodiment of the method of producing a Cannabis spp. Plant having a pathogen resistance trait of interest, the molecular markers may be for detecting polymorphisms at regular intervals within the pathogen resistance QTL such that recombination can be excluded. In an alternative embodiment, the molecular markers may be for detecting polymorphisms at regular intervals within the pathogen resistance QTL such that recombination can be quantified to estimate linkage disequilibrium between a particular polymorphism and the pathogen resistance phenotype. For example, molecular markers may be for detecting polymorphisms such that recombination events can be detected to a resolution of 10′000 or 100′000 or 500′000 base pairs within the QTL. It will be appreciated by those of skill in the art that several possible markers may be designed for detecting the polymorphisms. In one embodiment, the molecular markers may be designed based on a context sequence for the polymorphism described in Table 3 or 13, or may be selected from the primer pairs as defined in Table 4 or 11. In another embodiment, the molecular markers may further be designed based on a context sequence for a causative polymorphism described in Table 8 or a genetic marker linked to the QTL described in Table 13.

According to a further embodiment of the method of producing a Cannabis spp. plant having a pathogen resistance trait of interest, the pathogen resistance QTL may be an increased pathogen resistance QTL, a decreased pathogen resistance QTL, or an intermediate pathogen resistance QTL as defined in Table 2 or 9. Of particular use in producing a Cannabis spp. plant having a pathogen resistance trait of interest, are the polymorphisms selected from the group consisting of “common_3407”, “common_703”, “common_1691”, “common_701”, or combinations thereof, as defined in Table 2 or 9, which have been shown to have particularly high predictive value for the pathogen resistance QTL and trait.

According to a third aspect of the present invention there is provided for a method of producing a Cannabis spp. plant that has a pathogen resistance trait of interest, the method comprising introducing at least one pathogen resistance QTL characterized by one or more polymorphisms associated with the pathogen resistance trait of interest as defined in Table 2 or 9 into a Cannabis spp. plant, wherein said QTL is associated with the pathogen resistance trait of interest in the plant. In one embodiment, introducing the at least one pathogen resistance QTL comprises crossing a donor parent plant having the at least one pathogen resistance QTL with a recipient parent plant. In an alternative embodiment, introducing the at least one pathogen resistance QTL comprises genetically modifying the Cannabis spp. plant. Several methods of genetic modification are known to those of skill in the art, including targeted mutagenesis, genome editing, and gene transfer. For example, one or more of the polymorphisms associated with the pathogen resistance trait of interest as defined in Table 2 or 9 herein, or a causative polymorphism linked thereto, may be introduced into a plant by mutagenesis and/or gene editing. In particular the methods of genetically modifying a plant may be selected from the group consisting of CRISPR-Cas9 targeted gene editing, heterologous gene expression using various expression cassettes; TILLING, and non-targeted chemical mutagenesis using e.g., EMS. For example, CRISPR-Cas9 targeted gene editing may be achieved using a guide RNA. Alternatively, a cannabis spp. plant may be transformed with a cassette containing the QTL associated with the pathogen resistance trait of interest or a part thereof, via any transformation method known in the art.

In a one embodiment of the method of producing a Cannabis spp. plant that has a pathogen resistance trait of interest, the pathogen resistance QTL may be selected from the group consisting of:
i. a quantitative trait locus having a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL, such as a genetic marker described in Table 13;
ii. a quantitative trait locus having a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL;
iii. a quantitative trait locus defined by a single nucleotide polymorphism at position 79997 of NC_044373.1 of the CS10 genome and associated with pathogen resistance as defined in Table 2, or a genetic marker linked to the QTL; and/or
iv. a combination of any of (i) to (iii).

According to a further embodiment of the method of producing a Cannabis spp. plant that has a pathogen resistance trait of interest, the pathogen resistance trait may be a Botrytis cinerea resistance trait.

According to a fourth aspect of the present invention there is provided for a Cannabis spp. plant characterized according to the method for characterizing a Cannabis spp. plant with respect to a pathogen resistance phenotype as described herein.

In a fifth aspect of the present invention there is provided for a Cannabis spp. plant produced according to the method of a producing a Cannabis spp. plant having a pathogen resistance trait of interest as described herein. In some embodiments, the Cannabis spp. plant characterized or produced according to the method as described herein is not exclusively obtained by means of an essentially biological process.

According to a further aspect of the present invention there is provided for a Cannabis spp. plant comprising at least one pathogen resistance QTL characterized by one or more polymorphisms associated with a pathogen resistance trait of interest as defined in Table 2 or 9. In some embodiments, the plant is not exclusively obtained by means of an essentially biological process.

In one embodiment of the Cannabis spp. Plant of the invention, the pathogen resistance trait is a Botrytis cinerea resistance trait.

According to another aspect of the present invention there is provided for a quantitative trait locus that controls a pathogen resistance trait in Cannabis spp., wherein the quantitative trait locus has a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL, such as a genetic marker described in Table 13.

According to another aspect of the present invention there is provided for a quantitative trait locus that controls a pathogen resistance trait in Cannabis spp., wherein the quantitative trait locus has a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL.

In yet a further aspect of the present invention there is provided for a quantitative trait locus that controls a pathogen resistance trait in Cannabis spp., wherein the quantitative trait locus is defined by a single nucleotide polymorphism at position 79997 of NC_044373.1 of the CS10 genome and is associated with pathogen resistance as defined in Table 2, or a genetic marker linked to the QTL. In some embodiments, the quantitative trait loci may be comprised on an isolated nucleic acid.

According to one aspect of the present invention there is provided for a Cannabis spp. plant comprising a quantitative trait locus, or an isolated nucleic acid comprising the quantitative trait locus, defined herein.

In particular, there is provided for a Cannabis spp. plant comprising a pathogen resistance QTL selected from the group consisting of:
i. a quantitative trait locus that controls a pathogen resistance trait in Cannabis spp., wherein the quantitative trait locus has a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL, such as a genetic marker described in Table 13;
ii. a quantitative trait locus that controls a pathogen resistance trait in Cannabis spp., wherein the quantitative trait locus has a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL; and
iii. a quantitative trait locus that controls a pathogen resistance trait in Cannabis spp., wherein the quantitative trait locus is defined by a single nucleotide polymorphism at position 79997 of NC_044373.1 of the CS10 genome and is associated with pathogen resistance as defined in Table 2, or a genetic marker linked to the QTL; and/or
iv. a combination of any of (i) to (iii).

In a further aspect there is provided for an isolated gene that controls a pathogen resistance trait in a Cannabis spp. plant, wherein the gene is selected from the group consisting of the genes as defined in Table 7 with reference to the CS10 genome. In particular, the pathogen resistance trait may be a Botrytis cinerea resistance trait.

According to a further aspect there is also provided for possible causative polymorphisms for botrytis resistance in a cannabis spp. plant selected from: a polymorphism in a gene encoding a Protein kinase domain-containing protein (PKD) (NCBI accession: XP_030492442.1) at position 23309645 on chromosome NC_044371.1 with reference to the CS10 genome; and a polymorphism in a gene encoding a PTI6-like protein (NCBI accession: XP_030505675.1) at position 86872263 on chromosome NC_044375.1, as described in Table 8.

### BRIEF DESCRIPTION OF THE FIGURES

Non-limiting embodiments of the invention will now be described by way of example only and with reference to the following figures:

FIG. 1: Segregation of Botrytis resistance in the F2 populations tested. The F2 population designation is shown above each plot. The Y-axis shows plant count and the X-axis show the resistance score from 1-9 with 9 being most susceptible.

FIG. 2: A Manhattan plot showing the results of a GWA of Botrytis resistance of a combined Cannabis F2 population using Mixed Linear Model. Each box represents a separate chromosome, with the chromosome name above the plot and the position (in base pairs) on the chromosome on the X-axis below, the Y-axis is the LOD score, −log 10(p).

FIG. 3: A Manhattan plot showing the results of a GWA of Botrytis resistance of a training population of diverse cannabis genotypes using Mixed Linear Model. Each box represents a separate chromosome, with the chromosome name above the plot and the position (in base pairs) on the chromosome on the X-axis below, the Y-axis is the LOD score, −log 10(p).

FIG. 4: A multiple regression model of Botrytis resistance comparing random markers to markers identified in QTL1.

### SEQUENCES

The nucleic acid and amino acid sequences listed herein and in any accompanying sequence listing are shown using standard letter abbreviations for nucleotide bases, and the standard one or three letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

Methods are provided herein for identifying and obtaining plants having a pathogen resistance trait of interest, preferably a Botrytis resistance trait of interest, using a molecular marker detection technique. The inventors of the present invention have further produced and selected for increased Botrytis resistance in cannabis plants by crossing plants with various degrees of the botrytis resistance trait. Also demonstrated herein, the inventors were able to use genome wide association (GWA) to identify multiple QTLs linked to increased Botrytis resistance and to show that these QTLs contribute in combination to Botrytis resistance. The method provides evidence that the genetic basis of Botrytis resistance is polygenic. The inventors also show that the QTLs identified for Botrytis resistance can be extended to providing general pathogen resistance to a range of plant pathogens as well. This finding provides for the improvement of methods for producing plants displaying differing degrees of botrytis resistance.

A total of three QTLs for Botrytis resistance were identified in the combined F2 populations tested.

Tables 2 and 9 herein provide several single nucleotide polymorphisms (SNPs) which define the QTLs associated with a pathogen resistance trait of interest, specifically a Botrytis resistance trait of interest. In some embodiments one or more of the identified SNPs can be used to incorporate the increased pathogen resistance trait from a donor plant, containing one or more of the QTLs associated with the trait, into a recipient plant. For example, the incorporation of the pathogen resistance phenotype may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying one or more QTLs that are characterized by a haplotype comprising of a series of polymorphisms in linkage disequilibrium are provided. The QTLs each display limited frequency of recombination within the QTLs. Preferably the polymorphisms are selected from any one described in Table 2 or 9 herein, representing the pathogen resistance QTLs, or a genetic marker linked thereto, such as a genetic marker described in Table 13. Molecular markers may be designed for use in detecting the presence of the polymorphisms and thus the QTLs. Further, the identified QTL polymorphisms and the associated molecular markers may be used in a cannabis breeding program to predict the pathogen resistance trait of interest of plants in a breeding population and can be used to produce cannabis plants that either display an increased pathogen resistance trait, or do not, compared to a control population.

As used herein, reference to a plant or a variety with a “pathogen resistance trait” refers to a plant or a variety that shows resistance to infection by, and disease symptoms from a broad array of plant pathogen species, in particular the fungal species Botrytis cinerea, during the course of plant growth, at the time of harvest, and in post-harvest.

As used herein, reference to a plant or a variety with a “general resistance” refers to a plant or a variety that show resistance to infection by, and disease symptoms from, a broad array of pathogen species during the course of plant growth, at the time of harvest, and in post-harvest. General resistance may represent general defences that limit the spread of pathogens through plant tissues.

As used herein, reference to a plant or variety with a “Botrytis resistance trait” refers to plant or variety that shows resistance to infection by, and disease symptoms from, the fungal species Botrytis cinerea during the course of plant growth, at the time of harvest and in post-harvest.

A “pathogen resistance trait of interest” refers to the state of the plant with respect to the pathogen resistance trait, and includes increased pathogen resistance, decreased pathogen resistance, or an intermediate level of pathogen resistance compared with a control population.

As used herein, reference to a plant or variety with an “increased pathogen resistance trait” refers to a plant or variety having a propensity for increased pathogen resistance compared to plants from which it is derived. In some cases, a plant or variety with an increased pathogen resistance trait has a propensity for increased pathogen resistance in comparison to the mean pathogen resistance of plants from a population from which the plant or variety was derived.

As used herein, reference to a plant or variety with a “decreased pathogen resistance trait” refers to a plant or variety having a propensity for decreased pathogen resistance compared to plants from which it is derived. In some cases, a plant or variety with an decreased pathogen resistance trait has a propensity for decreased pathogen resistance in comparison to the mean pathogen resistance of plants from a population from which the plant or variety was derived.

As used herein, reference to a plant or variety with an “intermediate pathogen resistance trait” refers to a plant or variety having a propensity for intermediate pathogen resistance compared to plants from which it is derived. In some cases, a plant or variety with an intermediate pathogen resistance trait has a propensity for intermediate or average pathogen resistance in comparison to the mean pathogen resistance of plants from a population from which the plant or variety was derived.

The “time of harvest” is defined with respect to the maturity of the flower, where approximately greater than 50% of the pistils have turned brown in appearance. The “time of harvest” can also be determined by initiation of flowering for hemp-type cannabis or by other agronomic criteria common in the art.

It is a particular aim of the present invention to identify and characterize a plant for the pathogen resistance trait of interest early in the plant lifecycle, particularly prior to the plant displaying the pathogen resistance trait of interest, more particularly to incorporate the pathogen resistance trait of interest into the breeding population early on. This can be achieved by genotyping the plant using molecular markers for detecting at least one QTL associated with the pathogen resistance trait of interest prior to the time of harvest.

As used herein a “quantitative trait locus” or “QTL” is a polymorphic genetic locus with at least two alleles that differentially affect the expression of a continuously varying phenotypic trait when present in a plant or organism which is characterised by a series of polymorphisms in linkage disequilibrium with each other.

As used herein, the term “pathogen resistance QTL” or “pathogen resistance quantitative trait locus” refers to a quantitative trait locus comprising part, or all, of the QTLs characterized by the polymorphisms having an allelic state associated with pathogen resistance, and in particular Botrytis resistance, described Table 2 or 9, or characterized by combinations of said polymorphisms.

In some cases, it is particularly desirable to obtain a plant displaying a pathogen resistance trait or botrytis resistance trait of interest, where it is desired to obtain a plant displaying an increased pathogen resistance trait. However, resistance traits may impart a growth penalty on plant development, which can include, among others, an impact on plant growth, floral development, and yield of desirable cannabis products including flower, fiber, and seed. Thus, in some cases it may be more desirable to obtain plants displaying a decreased pathogen resistance trait, or an intermediate pathogen resistance trait. Thus, the pathogen resistance QTL may be an increased pathogen resistance QTL, a decreased pathogen resistance QTL, or an intermediate pathogen resistance QTL as defined herein.

As used herein, the term “increased pathogen resistance QTL” or “increased pathogen resistance quantitative trait locus” refers to a quantitative trait locus characterized by one or more polymorphisms having an allelic state associated with increased pathogen resistance described or defined in Table 2 or 9.

As used herein, the term “decreased pathogen resistance QTL” or “decreased pathogen resistance quantitative trait locus” refers to a quantitative trait locus characterized by one or more polymorphisms having an allelic state associated with decreased pathogen resistance described or defined in Table 2 or 9.

As used herein, the term “intermediate pathogen resistance QTL” or “intermediate pathogen resistance quantitative trait locus” refers to a quantitative trait locus characterized by one or more polymorphisms having an allelic state associated with intermediate pathogen resistance described or defined in Table 2 or 9.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “pathogen resistance haplotype” refers to the subset of the polymorphisms contained within any one of the pathogen resistance QTLs which exist on a single haploid genome complement of the diploid genome, and which are in linkage disequilibrium with the pathogen resistance trait. Exemplary polymorphisms for determining the pathogen resistance haplotype are provided in Tables 2, 9 and 13 herein.

As used herein, the term “donor parent plant” refers to a plant having a pathogen resistance haplotype, or one or more pathogen resistance alleles, associated with the pathogen resistance trait of interest.

As used herein, the term “recipient parent plant” refers to a plant having a pathogen resistance haplotype, or one or more pathogen resistance alleles, not associated with the pathogen resistance trait of interest.

The term “pathogen resistance allele” refers to the haplotype allele within a particular QTL that confers, or contributes to, the pathogen resistance trait of interest, or alternatively, is an allele that allows the identification of plants with the pathogen resistance trait of interest that can be included in a breeding program, particularly to select for the pathogen resistance trait (“marker assisted breeding”, “marker assisted selection”, or “genomic selection”).

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the pathogen resistance trait, in particular the Botrytis resistance trait.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, and gene duplications.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the pathogen resistance trait. In the context of association studies, the genetic variants which are responsible for the association signal at a locus are referred to as the “causal variants”. Causal variants may comprise one or more “causal polymorphisms” that have a biological effect on the phenotype.

The term “nucleic acid” encompasses both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTLs of the invention or genes identified herein, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid(s) or gene(s) identified herein may be isolated nucleic acids or gene(s), which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids, and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs10 reference genome” or “cs10 genome”.

### Methods of Identifying a QTL or Haplotype Responsible for Pathogen Resistance, Particularly Botrytis Resistance, and Molecular Markers Therefor

In some embodiments, methods are provided for identifying a QTL or haplotype responsible for pathogen resistance and for selecting plants with the pathogen resistance trait of interest, thereby to identify the QTL or haplotype responsible for the trait. In some embodiments, the methods may comprise the steps of:

a. Identifying a plant that displays the pathogen resistance phenotype within a breeding program.

b. Establishing a population by crossing the identified plant to itself (selfing) or a recipient parent plant.

c. Genotyping the resultant F1, or subsequent populations, for example by sequencing methods.

d. Performing association studies, including phenotyping and linkage analysis, to discover QTLs and/or polymorphisms contained within the QTL.

e. Optionally, identifying cannabis paralogs of previously characterized genes that may be involved in conferring the pathogen resistance trait.

f. Developing molecular markers that detect one or more polymorphisms linked to QTLs, alleles within these QTLs, or existing or induced polymorphisms.

g. Validating the molecular markers by determining the linkage disequilibrium between the marker and the pathogen resistance trait.

### Trait Development and Introgression

In some embodiments, methods are provided for marker assisted breeding (MAB) or marker assisted selection (MAS) of plants having a pathogen resistance QTL or displaying the pathogen resistance trait. The methods may comprise the steps of:

a. Identifying a plant that displays the pathogen resistance trait or phenotype or contain a pathogen resistance QTL as defined herein.

b. Establishing a population by crossing the identified plant to itself (selfing) or another recipient parent plant.

c. Genotyping and phenotyping the resultant F1, or subsequent, populations, for example by sequencing methods.

d. Performing association studies, inputting phenotype and genotype information to identify genomic regions enriched with polymorphisms associated with the pathogen resistance trait, to discover QTLs and/or polymorphisms contained within the QTL.

e. Optionally, identifying cannabis paralogs of previously characterized genes that may be involved in the pathogen resistance trait.

f. Developing molecular markers that detect one or more polymorphisms linked to QTLs, alleles within these QTLs, or existing or induced polymorphisms.

g. Using the molecular markers when introgressing the QTLs or polymorphisms into new or existing cannabis varieties to select plants containing the pathogen resistance haplotype or the pathogen resistance trait, or plants where the pathogen resistance haplotype or the pathogen resistance trait is absent.

### QTLs and Marker Assisted Breeding

In some embodiments, during the breeding process, selection of plants displaying the pathogen resistance may be based on molecular markers designed to detect polymorphisms linked to genomic regions that control the trait of interest by either an identified or an unidentified mechanism. Previously identified genetic mechanisms may, for example, have a direct or pleiotropic effect on pathogen and/or botrytis resistance in a plant. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the pathogen resistance phenotype, based on identification of polymorphisms that are either linked to, or found within QTLs that are associated with pathogen resistance phenotype using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the pathogen resistance phenotype are identified within synthetic populations of plants capable of revealing dominant, recessive, or complex traits. In one embodiment of the invention, a genetically diverse population of cannabis varieties, that are used to produce the synthetic population are integrate them into a breeding program by unnatural processes. In some embodiments, the identification of parental plants that are a source of resistance are used to create F2 populations. Specific parental populations may be the source of general and/or novel traits. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the following methods are employed to integrate the plants into a breeding program include some or all of the following:
a. Growing plants in rich media or soils under artificial lighting;
b. Cloning of plants, often through a multitude of sub-cloning cycles;
c. Introduction of plants into in vitro, sterile growth environments, and subsequent removal to standard growth conditions;
d. Exposure to mutagens such as EMS, colchicine, silver nitrate, ethidium bromide, dinitroanalines, high concentrations mono or poly-chromatic light sources;
e. Growing plants under highly stressful conditions which include restricted space, drought, pathogen challenge, atypical temperatures, and nutrient stresses.

### Pathogen Resistance Trait of Interest Association Studies and QTL Identification

In some embodiments, the synthetic populations created are either the offspring of sexual reproduction or clones of plants in the breeding program such that genetic material of individuals in the synthetic populations is derived from one, or two, or more plants from the breeding program.

In one embodiment, plants identified within the synthetic population as having a trait of interest, such as the botrytis resistance trait, may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype. In this case the trait is the pathogen resistance phenotype, in particular the Botrytis resistance phenotype. In a population generated by crossing, the amount of linkage disequilibrium (LD) is reduced between genetic marker and the QTL as a function of genetic distance in cannabis varieties with similar genome structures. Simple association mapping is performed by biparental crosses of two closely related lines where one line has a phenotype of interest and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, the pathogen resistance phenotype or botrytis resistance phenotype of the plants. Using the association studies described herein, together with accurate phenotyping, this method is able to identify genomic regions, QTLs and even specific genes or polymorphisms responsible for the pathogen resistance or botrytis resistance phenotype that are directly introduced into recipient lines. Polygenic phenotypes may also be identified using the methods described herein.

In one embodiment, the structured population is grown to the flowering stage. To characterize the phenotypes of the lines they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can improve the accuracy of genomic selection models. In some embodiments, a genomic selection model that incorporates pathogen resistance can be improved by the inclusion of the pathogen resistance QTLs in the GS model. In some embodiments, the SNPs described in Tables 2, 9 and 13 herein may be useful in a genomic selection model, for example where genotypes with unknown phenotypes are evaluated using an approach like a random forest algorithm for prediction of the pathogen resistance trait, and particularly in combination, to improve the predictive power of the model. In a further embodiment, combinatorial markers for strengthening genomic selection are provided in Tables 5, 6 and 12.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system, including primers designed based on the context sequences for the polymorphisms identified herein, provided in Table 3, 10 or 13. Such molecular markers may include PCR primers, or targeted sequencing primers such as those described in the examples below, more specifically the primers defined in Table 4 or 11.

For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two or more of the SNPs in the haplotype can be used to identify the presence of the QTL and by association, the pathogen resistance trait of interest.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the pathogen resistance trait of interest.

According to some embodiments, any polymorphism in linkage disequilibrium with one or more of the pathogen resistance QTLs can be used to determine the pathogen resistance haplotype in a breeding population of plants, as long as the polymorphism is unique to the pathogen resistance trait in the donor parent plant when compared to the recipient parent plant.

In some embodiments the desired trait is the increased pathogen resistance trait, and the donor parent plant may be a plant that has been genetically modified or selected to include an increased pathogen resistance QTL defined by a polymorphism associated with the increased pathogen resistance trait, for example any, some, or all of the polymorphisms defined in Table 2 or 9 associated with the trait.

Alternatively, the desired trait may be the decreased pathogen resistance trait, or intermediate pathogen resistance trait, and the donor parent plant may be a plant that has been genetically modified or selected to include a decreased pathogen resistance QTL or intermediate pathogen resistance QTL defined by a polymorphism associated with the decreased pathogen resistance trait or the intermediate pathogen resistance trait, for example any, some, or all of the polymorphisms defined in Table 2 or 9 associated with the trait, or combinations thereof as described herein.

In some embodiments, donor parent plants, as described above, are used as one of two parents to create breeding populations (F1) through sexual reproduction. In this embodiment, donor parent plants may be identified by detecting polymorphisms using the molecular markers as described above.

Methods for reproduction that are known in the art may be used. The donor parent plant provides the trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the pathogen resistance allele or pathogen resistance haplotype in plants to be used in the F1 cross is determined using the described molecular markers. In some embodiments, the resulting F2 progeny, or subsequent progeny, is/are screened for any of the pathogen resistance polymorphisms, and in particular Botrytis resistance polymorphisms, described herein.

The plants at any generation can be produced by asexual means like cutting and cloning, or any method that yields a genetically identical offspring.

Production of Cannabis Spp. Plants Having the Increased Pathogen Resistance Trait

It is a particular aim of the present invention to provide for the production of Cannabis spp. plants that have an increased pathogen resistance trait. Accordingly, in some embodiments, a Cannabis spp. plant that has the decreased pathogen resistance trait may be converted into a plant having an increased pathogen resistance trait according to the methods of the present invention by providing a breeding population where the donor parent plant contains an increased pathogen resistance QTL associated with an increased pathogen resistance trait and the recipient parent plant either displays the decreased pathogen resistance trait or contains the decreased pathogen resistance QTL.

In some embodiments the decreased pathogen resistance phenotype may be removed from a recipient parent plant by crossing it with a donor parent plant having the increased pathogen resistance QTL. In some embodiments the donor parent plant has an increased pathogen resistance phenotype and a contains a contiguous genomic sequence characterized by one or more of the polymorphisms of Table 2 or 9 associated with the increased pathogen resistance allele or a haplotype conferring the increased pathogen resistance trait.

In some embodiments, the donor parent plant is any cannabis spp. variety that is cross fertile with the recipient parent plant.

In some embodiments, MAS or MAB may be used in a method of backcrossing plants carrying the pathogen resistance trait, in particular the Botrytis resistance trait, to a recipient parent plant. For example, an F1 plant from a breeding population can be crossed again to the recipient parent plant. In some embodiments, this method is repeated.

In some embodiments, the resulting plant population is then screened for the pathogen resistance trait using MAS with molecular markers to identify progeny plants that contain one or more polymorphism, such as any of those described Table 2 or 9, indicating the presence of an allele of a QTL associated with the increased pathogen resistance phenotype. In another embodiment, the population of cannabis plants may be screened by any analytical methods known in the art to identify plants with desired characteristics, specifically increased pathogen resistance.

Production of Cannabis Spp. Plants Having Decreased- or Intermediate Pathogen Resistance

In some embodiments, a Cannabis spp. plant that has the increased pathogen resistance trait may be converted into a plant having a decreased pathogen resistance trait or intermediate pathogen resistance trait according to the methods of the present invention by providing a breeding population where the donor parent plant contains decreased pathogen resistance QTL associated with a decreased pathogen resistance trait, or an intermediate pathogen resistance QTL associated with the intermediate pathogen resistance trait, and the recipient parent plant either displays the increased pathogen resistance phenotype or contains the increased pathogen resistance QTL.

In some embodiments the increased pathogen resistance phenotype may be removed from a recipient parent plant by crossing it with a donor parent plant having the decreased- or intermediate pathogen resistance QTL. In some embodiments the donor parent plant has a decreased- or intermediate pathogen resistance phenotype and a contains a contiguous genomic sequence characterized by one or more of the polymorphisms of Table 2 or 9 associated with the decreased- or intermediate pathogen resistance allele or haplotype associated therewith.

In some embodiments, the donor parent plant is any cannabis spp. Variety that is cross fertile with the recipient parent plant.

In some embodiments, MAS or MAB may be used in a method of backcrossing plants carrying the decreased- or intermediate pathogen resistance trait to a recipient parent plant. For example, an F1 plant from a breeding population can be crossed again to the recipient parent plant. In some embodiments, this method is repeated.

In some embodiments, the resulting plant population is then screened for the pathogen resistance trait using MAS with molecular markers to identify progeny plants that contain one or more polymorphism, such as any of those described Table 2 or 9, indicating the presence of an allele of a QTL associated with the decreased- or intermediate pathogen resistance phenotype. In another embodiment, the population of cannabis plants may be screened by any analytical methods known in the art to identify plants with desired characteristics, specifically the decreased- or intermediate pathogen resistance trait.

### Methods to Genetically Engineer Plants to Achieve the Increased Pathogen Resistance or Decreased Pathogen Resistance Using Mutagenesis or Gene Editing Techniques

Identifying QTLs, and individual polymorphisms, that correlate with a trait when measured in an F1, F2, or similar, breeding population indicates the presence of one or more causative polymorphisms in close proximity the polymorphism detected by the molecular marker. In some embodiments, the polymorphisms associated with the increased-, decreased-, or intermediate pathogen resistance trait are introduced into a plant by other means so that a trait, such as the pathogen resistance trait, can be introduced into plants that would not otherwise contain associated causative polymorphisms or removed from plants that would otherwise contain associated causative polymorphisms. For example, the polymorphisms detailed in Tables 2 and 9 are molecular markers that can be used to indicate the presence of a possible causative polymorphism. Further, the polymorphisms detailed in Table 13 may also be useful as molecular markers for predicting the presence of possible causative polymorphisms.

The entire QTLs or parts thereof which confer the pathogen resistance trait of interest described herein may be introduced into the genome of a cannabis spp. plant to obtain plants with the pathogen resistance trait of interest through a process of genetic modification known in the art, for example, but not limited to, heterologous gene expression using an expression cassette including a sequence encoding the QTL(s) or part thereof, the gene(s), or the nucleic acids comprising them. The expression cassettes may contain all or part of the QTL(s) or gene(s), including possible causative polymorphisms.

The trait described herein may be removed from, or introduced into, the genome of a cannabis spp. plant to obtain plants that exclude or include the causative polymorphisms and the potential to display a desired pathogen resistance trait of interest through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with the pathogen resistance of interest as described in Table 2 or 9 or a causative polymorphism linked thereto. Specific causative polymorphisms may include a polymorphism in a gene encoding a Protein kinase domain-containing protein (PKD) (NCBI accession: XP_030492442.1) at position 23309645 on chromosome NC_044371.1 with reference to the CS10 genome; and a polymorphism in a gene encoding a PTI6-like protein (NCBI accession: XP_030505675.1) at position 86872263 on chromosome NC_044375.1, as described in Table 8. Non limiting examples of SSNs include zinc finger nucleases (ZFNs), TAL effector nucleases (TALENs), meganuclease, and, clustered regularly interspaced short palindromic repeats (CRISPR)/CRISPR-associated protein (Cas) system. In some embodiments, non-limiting examples of Cas proteins suitable for use in the methods of the present invention include Csnl, Cpf1 Cas9, Cas 12, Cas 13, Cas 14, CasX and combinations thereof. In one embodiment, a modified Cannabis spp. plant having a pathogen resistance trait of interest, such as a Botrytis resistance trait, is generated using CRISPR/Cas9 technology, which is based on the Cas9 DNA nuclease guided to a specific DNA target by a single guide RNA (sgRNA). For example, the genome modification may be introduced using guide RNA, e.g., single guide RNA (sgRNA) designed and targeted to introduce a polymorphism associated with the pathogen resistance trait of interest, such as a polymorphism associated with the pathogen resistance trait of interest described in Table 2 or 9, or a causative polymorphism linked thereto, such as a polymorphism in a gene encoding a Protein kinase domain-containing protein (PKD) (NCBI accession: XP_030492442.1) at position 23309645 on chromosome NC_044371.1 with reference to the CS10 genome; and a polymorphism in a gene encoding a PTI6-like protein (NCBI accession: XP_030505675.1) at position 86872263 on chromosome NC_044375.1, as described in Table 8.

DNA introduction into the plant cells can be performed using Agrobacterium infiltration, virus-based plasmid delivery of the genome editing molecules and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g., Cas9, Cpf 1) with a predefined guide RNA molecule (gRNA). The gRNA is complementary to a specific DNA sequence targeted for editing in the plant genome and which guides the Cas nuclease to a specific nucleotide sequence. The gRNA may be designed based on the context sequence for the polymorphisms provided in Table 3, 8, 10 or 13. The predefined gene specific gRNA's may be cloned into the same plasmid as the Cas gene and this plasmid is inserted into plant cells as described above.

In some embodiments, once the guide RNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion the gene coding sequence for a gene responsible for botrytis susceptibility is disrupted and the translation of the encoded protein is compromised either by a premature stop codon or disruption of a functional or structural property of the protein.

In another embodiment, the pathogen resistance trait of interest in Cannabis spp. plants may be introduced by generating gRNA with homology to a specific site of predetermined genes in the Cannabis genome or the QTLs defined herein. This gRNA may be sub-cloned into a plasmid containing the Cas9 gene, and the plasmid inserted into the Cannabis spp. plant cells. In this way site specific mutations in the QTLs are generated, particularly causative polymorphisms linked to a SNP associated with the pathogen resistance trait of interest described in Table 2, 9 or 13, thus effectively conferring pathogen resistance in the genome edited plant. In some embodiments, the causative polymorphism may be a polymorphism in a gene encoding a Protein kinase domain-containing protein (PKD) (NCBI accession: XP_030492442.1) at position 23309645 on chromosome NC_044371.1 with reference to the CS10 genome; and a polymorphism in a gene encoding a PTI6-like protein (NCBI accession: XP_030505675.1) at position 86872263 on chromosome NC_044375.1, as described in Table 8.

In some embodiments, a modified Cannabis spp. plant exhibiting an increased pathogen resistance trait, such as increased botrytis resistance, may be obtained using the targeted genome modification methods described above, wherein the plant comprises a targeted genome modification to introduce one or more polymorphisms associated with the increased pathogen resistance trait, particularly a causative polymorphism linked to a SNP defined in Table 2, 9 or 13, or alternatively a causative polymorphism detailed in Table 8, wherein the modification effects an increased pathogen resistance trait.

In some embodiments, the genetic modification may be introduced using gene silencing, a process by which the expression of a specific gene product is lessened or attenuated. Gene silencing can take place by a variety of pathways, including by RNA interference (RNAi), an RNA dependent gene silencing process. In one embodiment, RNAi may be achieved by the introduction of small RNA molecules, including small interfering RNA (siRNA), microRNA (miRNA) or short hairpin RNA (shRNA), which act in concert with host proteins (e.g., the RNA induced silencing complex, RISC) to degrade messenger RNA (mRNA) in a sequence-dependent fashion. In particular, RNAi may be used to silence one or more of the putative causative genes described in Table 7 herein. Such RNAi molecules may be designed based on the sequence of these genes. These molecules can vary in length (generally 18-30 base pairs) and may contain varying degrees of complementarity to their target mRNA in the antisense strand. Some, but not all, RNAi molecules have unpaired overhanging bases on the 5′ or 3′ end of the sense strand and/or the antisense strand. As used herein, the term “RNAi molecule” includes duplexes of two separate strands, as well as single strands that can form hairpin structures comprising a duplex region. The RNAi molecules may be encoded by DNA contained in an expression cassette and incorporated into a vector. The vector may be introduced into a plant cell using Agrobacterium infiltration, virus-based plasmid delivery of the vector containing the expression cassette and/or mechanical insertion of the vector (PEG mediated DNA transformation, biolistics, etc.).

Plants may be screened with molecular markers as described herein to identify transgenic individuals with or without pathogen resistance or having an increased, decreased or intermediate resistance QTL or polymorphism(s), following the genetic modification.

In some embodiments, cannabis spp. plants having one or more of the polymorphisms of Table 2, 9 or 13 associated with the increased pathogen resistance, decreased pathogen resistance, or intermediate pathogen resistance QTLs or a causative polymorphism linked thereto are provided. Possible causative polymorphism include a polymorphism in a gene encoding a Protein kinase domain-containing protein (PKD) (NCBI accession: XP_030492442.1) at position 23309645 on chromosome NC_044371.1 with reference to the CS10 genome; and a polymorphism in a gene encoding a PTI6-like protein (NCBI accession: XP_030505675.1) at position 86872263 on chromosome NC_044375.1, as described in Table 8. The polymorphisms may be introduced, for example, by genetic engineering. In some embodiments the one or more polymorphisms associated with the pathogen resistance of interest or linked thereto are introduced into the plants by breeding, such as by MAS or MAB, for example as described herein.

The increased pathogen resistance, decreased pathogen resistance, or intermediate pathogen resistance QTLs described herein, or genes identified herein responsible for conferring an increased, decreased, or intermediate pathogen resistance trait, may be under the control of, or operably linked to, a promoter, for example an inducible promoter. Such QTLs or genes may be operably linked to the inducible promoter so as to induce or suppress the pathogen resistance trait or phenotype in the plant or plant cell.

Accordingly, in a further embodiment, Cannabis spp. plants comprising an increased, decreased, or intermediate pathogen resistance QTL described herein, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Genome-Wide Association Studies (GWAS) of Botrytis Resistance in Cannabis

During outdoor field trials in 2020 the inventors grew to harvest several hundred distinct cannabis HRT and hemp cultivars of diverse origin. The investigators scored all plants for resistance or susceptibility to Botrytis at the time of harvest. The investigators identified cannabis plants that were generally resistant to Botrytis and plants that were susceptible to Botrytis infection. Until this time there was no indication of the resistance status of any genotype of cannabis to Botrytis. The inventors generated crosses between susceptible and resistant cannabis genotypes identified in the 2020 field experiment in order to create F2 populations that segregate for resistance to Botrytis. The inventors evaluated the F2 populations in a field experiment in 2021 at the time of harvest for resistance or susceptibility to Botrytis cinerea.

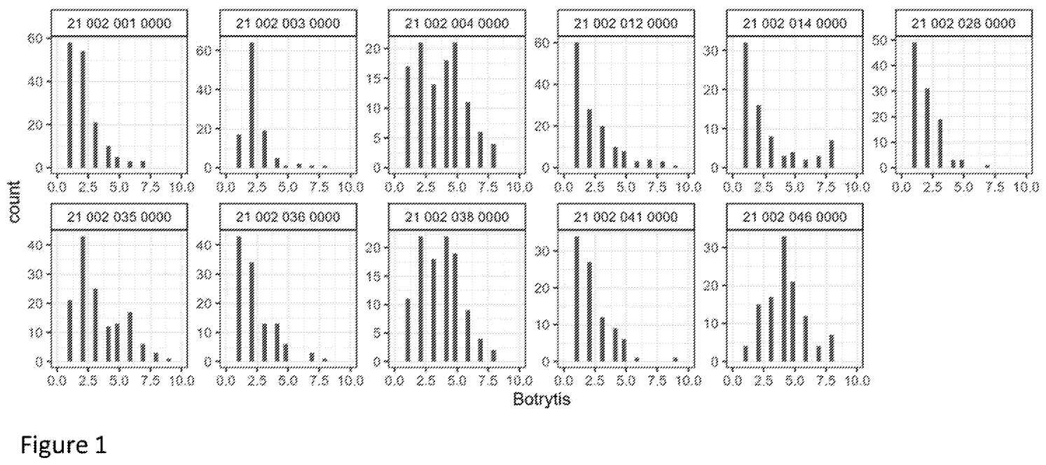

The inventors found that a number of the F2 populations generated had varying levels of Botrytis resistance amongst individuals at the time of harvest in the 2021 field trial. They sought to better understand the molecular basis for disease resistance by scoring disease symptoms in these designed F2 populations, comprising in total 1075 individuals (Table 1). The inventors found that resistance to Botrytis was segregating within the individual populations selected (FIG. 1).

TABLE 1
An overview of the F2 populations used including the (Average)
average Botrytis resistance score (a score from 1-9, where
1 is resistant and 9 is susceptible, the (StDev) the standard
deviation of the Botrytis resistance score, and the population
size given by the number of plants.

| Population ID | Average | StDev | Population size |
|---|---|---|---|
| 21 002 001 0000 | 2.162337662 | 1.359886477 | 154 |
| 21 002 003 0000 | 2.309090909 | 1.163240133 | 110 |
| 21 002 004 0000 | 3.732142857 | 1.958957909 | 112 |
| 21 002 012 0000 | 2.445255474 | 1.858682756 | 137 |
| 21 002 014 0000 | 2.75 | 2.333095226 | 76 |
| 21 002 028 0000 | 1.887850467 | 1.12708593 | 107 |
| 21 002 035 0000 | 3.323943662 | 1.966201276 | 142 |
| 21 002 036 0000 | 2.309734513 | 1.529937242 | 113 |
| 21 002 038 0000 | 3.644859813 | 1.722505505 | 107 |
| 21 002 041 0000 | 2.277777778 | 1.476617588 | 90 |
| 21 002 046 0000 | 4.230088496 | 1.700886584 | 113 |

At the time of harvest whole plants including flower, leaf, and stem were scored for susceptibility to Botrytis cinerea. This trait has been quantified using a subjective scale from 1 to 9 where 1 indicates plants with high resistance to Botrytis cinerea, while 9 indicate plants with high susceptibility to Botrytis i.e. showing the presence of Botrytis disease symptoms and Botrytis conidiophore. Fungal-pathogens that mimic the disease symptoms of Botrytis cinerea cannot be ruled out, however visual identification of Botrytis is commonplace.

To complement visual identification, PCR based assays were used to demonstrate the pathogen observed was Botrytis cinerea. DNA was extracted from about 70 mg of leaf discs from all the plants evaluated using an adapted kit with “sbeadex” magnetic beads by LGC Genomics. Published primers to detect the Botrytis cinerea, Glyceraldehyde 3-phosphate dehydrogenase (Botr_G3PDH_Fw ATT GAC ATC GTC GCT GTC AAC GA (SEQ ID NO:1) and Botr_G3PDH_Rv ACC CCA CTC GTT GTC GTA CCA (SEQ ID NO:2)) and Heat Shock Protein 60 genes (Botr_HSP60_Fw CAA TTG AGA TTT GCC CAC AAG (SEQ ID NO:3) and Botr_HSP60_Rv GAT GGA TCC AGT GGT ACC GAG CAT (SEQ ID NO:4)) were used with an annealing temp 55° C. and 120 sec elongation time. The PCR based assay confirmed the visual identification of Botrytis in all samples analyzed.

Looking at the individual populations, the segregation of the Botrytis resistance trait can be observed (FIG. 1). Notably the populations have distributions of plants between resistant and susceptible, however the average resistance in the individual population differ, where some populations are on average more susceptible to Botrytis than others. For example, population GID 210020460000 is comprised of plants that are more susceptible to Botrytis on average than the others (FIG. 1). This may indicate that the individual populations are segregating for shared as well as distinct resistance traits.

Following scoring for Botrytis resistance, All F2 plants described in Table 1 were sequenced.

DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel was employed. The inventors designed the marker panel based on a pangenome comprising 13 genotypes in order to include SNP markers common to a broad diversity of HRT and hemp cannabis including the Cannabis Sativa CS10 reference genome. Targeted sequencing was carried out on the Ion Torrent system by Thermo Fisher Scientific. The primers for the SNPs identified are provided in Table 4, below. The library pool was loaded onto Ion 550 chips with Ion Chef and sequenced with Ion GeneStudio S5 Plus according to the manufacturer's instructions (Ion 550™ Kit from Thermo Fisher Scientific).

From a population of 9 combined F2 populations with a total of 1075 individuals, a genome-wide association study (GWAS) was performed to detect significant associations between genotypic information derived from targeted resequencing of the custom SNP marker panel described above and the susceptibility of Cannabis to Botrytis. The Botrytis resistance scores from 1-9, where 1 indicates most resistant plants, were used as an input for GWAS.

The genotypic matrix was filtered for SNPs having a minor allele frequency lower than 1%. The GWA was performed using GAPIT version 3 (Wang and Zhang, 2021) with five statistical models: General Linear Model (GLM), Mixed Linear Model (MLM), FarmCPU and Blink (model=c(“GLM”, “MLM”, “FarmCPU”, “Blink”). A quantile-quantile plot (QQ plot) was used to evaluate the statistical models. In all models we failed to detect SNPs with significant association with Botrytis resistance.

For better modelling the inventors reasoned that a high rate of missing values may be impacting the estimation of population structure and kinship among individuals. To solve this, the GWA with all F2 populations combined was performed again but instead used a SNP matrix that underwent a round of imputation for reducing the number of missing values. In order to reduce missing data in the genotype file, an imputation has been performed using the HapMap_imputation software (GitHub-mwylerCH/HapMap_Imputation). Briefly, the genotype file is converted to a hapmap format (comma separated, http://augustogarcia.me/statgen-esalq/Hapmap-and-VCF-formats-and-its-integration-with-onemap/#hapmap).

In a first step, HapMap_Imputation counts the occurrence of each nucleotide at every single genotyped position. The most common nucleotide is defined as major allele, the second is defined as minor allele. Missing genotyping information is excluded. In the case major and minor alleles occur at the same number, the nucleotide of the reference cs10 (available as GCF_900626175.1 on NCBI) is chosen as major allele. Subsequently, HapMap_Imputation sorts markers by position and parses the hapmap into the required fastPHASE (Scheet and Stephens, 2006) input format. Briefly, HapMap_Imputation splits the haplotypes into two separate rows, converts major and minor alleles into 0 and 1 respectively and produces temporary files for each chromosome.

During the third step, HapMap_Imputation downloads the latest fastPHASE version and runs the imputation using 8 cores in parallel. fastPHASE is run with ten random starts of the imputation algorithm. After imputation, HapMap_Imputation reverses the 0 and 1 coding into the major and minor nucleotide, respectively. Subsequently, the two haplotypes are combined, and the separate chromosomes are merged into a single file.

This combination of techniques resulted in 4461 SNP markers after filtering using 9 F2 populations made up of a total 978 plants. The GWA was performed using GAPIT version 3 (Wang and Zhang, 2021) with a Mixed Linear Model (MLM). SNPs surpassing a LOD (−log₁₀(p-value)) value of 3 were considered to have a significant association with trait variation.

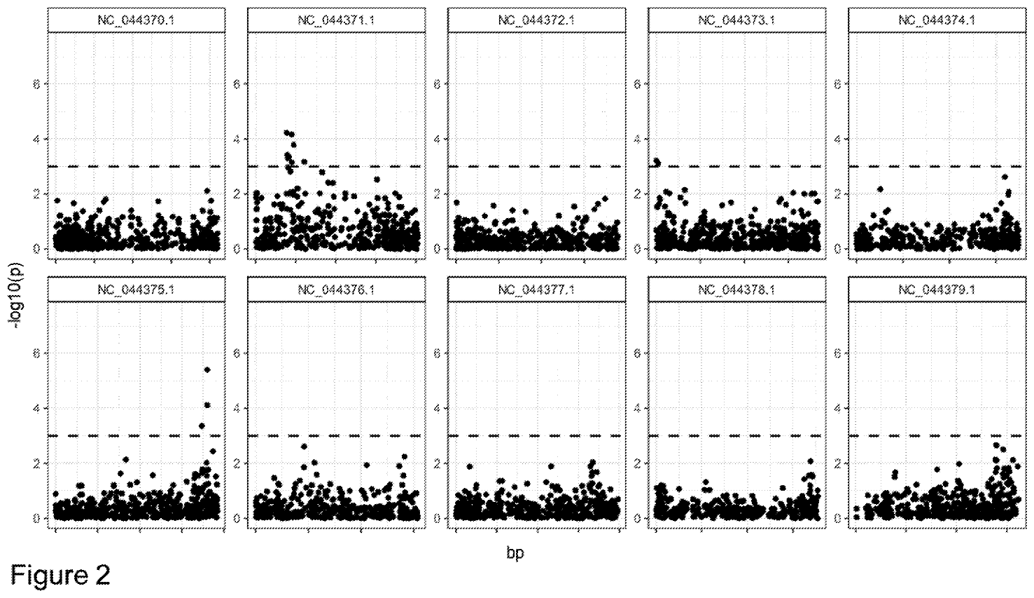

SNPs showing a significant association with susceptibility, with an LOD value greater than 3, were found on chromosome NC_044371.1, NC_044373.1, NC_044375.1 with reference to the Cannabis Sativa CS10 genome, shown in FIG. 2, defining three QTLs: QTL1, QTL2, and QTL3, respectively. The homozygous allele of the SNPs in Table 2 that can distinguish botrytis resistance are listed along with their position and reference sequence. Included also are the possible alleles at this position and the corresponding average resistance to Botrytis based on a score from 1-9, with a score of 1 being complete resistance. Interestingly, for each significant SNP identified as associated with Botrytis resistance, the heterozygous allele shows intermediate resistance compared to the homozygous alleles, indicating this may a semi-dominant trait.

Specifically, the inventors identified a QTL associated with Botrytis resistance on chromosome NC_044371.1—QTL1 at position 19201111-23661598 defined here by the identified SNPs in Table 2. The SNP most significantly associated with QTL1 is common_703, with an LOD score of 4.23. Based on these findings, if the presence of this or these SNPs associated with QTL are detected they can be used in marker assisted selection or marker assisted identification by someone skilled in the art improve the selection for Botrytis resistance by distinguishing plants with a propensity for Botrytis resistant form non-resistant plants and populations of cannabis (Table 2). In QTL1, when SNP common_703 is Allele_3, homozygous GG, this would indicate a plant with a significantly increased average resistance to Botrytis as compared to plants with the alternative allele, Allele_1, or that are heterozygous for this allele, Allele 2, with reference to Table 2.

A second locus associated with Botrytis resistance, QTL2, was identified on NC_044373.1 at position 79997 defined by SNP common_1691. Common_1691 can be similarly used in marker assisted selection or marker assisted identification by someone skilled in the art selectively to distinguish Botrytis resistant plants or populations from non-resistant plants. In QTL2 when SNP common_1691 is Allele 3, homozygous AA, this would indicate a plant with a significantly increased average resistance to Botrytis as compared to plants with the alternative allele, Allele_1, or that are heterozygous for this allele, Allele_1, with reference to Table 2.

The inventors identified a third locus associated with Botrytis resistance on chromosome NC_044375.1—QTL3 at position 86855033-90075725 defined here by the identified SNPs in Table 2. The SNP most significantly associated with QTL3 common_3407, with an LOD score of 5.44, can be used for marker assisted selection by someone skilled in the art to selectively distinguish Botrytis resistant from non-resistant plants and populations of cannabis, Table 2. In QTL3 when SNP common_3407 is Allele 3, homozygous AA, this would indicate a plant with a significantly increased average resistance to Botrytis as compared to plants with the alternative allele, Allele_2, or that are heterozygous for this allele, Allele_1, with reference to Table 2. We find that the multiple QTLs identified for Botrytis resistance provide evidence that the genetic basis of this trait is polygenic. The polygenic nature of this trait underlies why breeding for resistance can be challenging.

The reference sequence for each of the SNPs identified is given in Table 3 with reference to the CS10 genome. In Table 4, PCR primers designed to amplify each of the regions containing these SNPs, with reference to the CS10 genome, are provided in order for the allelic variant to be determined.

TABLE 2
SNPs associated with the Botrytis resistance in the F2 populations.
The presence of the resistance to Botrytis is predicted by the occurrence of
the indicative allele (marked with *). The positions and chromosome of the SNPs
are provided with reference to the CS10 reference genome as described herein.
The LOD score is provided as LOD. “Mean_1” denotes the average phenotypic
value associated with Allele_1 based on scoring for resistance from 1 to 9,
where 1 is the highest resistance, “Mean_2” denotes the average phenotypic
value associated with Allele_2 based on scoring for resistance from 1 to 9,
where 1 indicates the highest resistance and Mean_3 denotes the average
phenotypic value associated with Allele_3 scoring for resistance from 1 to 9,
where 1 indicates the highest resistance. Count_1, Count_2, and Count_3 denote
the number of plants that contributed to the average phenotypic value of Mean_1,
Mean_2, and Mean_3 respectively. The SNP position on the chromosome
is provided with reference to the CS10 reference genome.

| SNP NAME | Chromosome | Position | LOD | Allele_1 | Allele_2 | Allele_3 |
|---|---|---|---|---|---|---|
| common_3407 | NC_044375.1 | 90000409 | 5.4032377 | AC | CC | AA* |
| common_703 | NC_044371.1 | 19201111 | 4.2283093 | AA | AG | GG* |
| common_719 | NC_044371.1 | 22470941 | 4.1609971 | AA* | AT | TT |
| common_3408 | NC_044375.1 | 90075725 | 4.1137593 | AA* | CC | AC |
| GBScompat_{—} | NC_044371.1 | 23661598 | 3.7856471 | AG | GG | AA* |
| common_137 | | | | | | |
| GBScompat_{—} | NC_044371.1 | 19677542 | 3.4295348 | CC | GG* | CG |
| common_132 | | | | | | |
| common_3378 | NC_044375.1 | 86855033 | 3.3613306 | AA | AG | GG* |
| common_710 | NC_044371.1 | 20668886 | 3.3384756 | GG | AA* | AG |
| common_708 | NC_044371.1 | 19987181 | 3.300423 | AA* | AG | GG |
| common_1691 | NC_044373.1 | 79997 | 3.2155328 | AG | GG | AA* |
| common_766 | NC_044371.1 | 30275097 | 3.1667424 | AG | GG* | AA |

| SNP NAME | Mean_1 | Mean_2 | Mean_3 | Count_1 | Count_2 | Count_3 |
|---|---|---|---|---|---|---|
| common_3407 | 2.5611354 | 3.5884354 | 2.1806167 | 458 | 294 | 227 |
| common_703 | 3.4924731 | 2.3333333 | 1.8509615 | 465 | 306 | 208 |
| common_719 | 1.6860465 | 2.3396825 | 3.1851211 | 86 | 315 | 578 |
| common_3408 | 1.9466667 | 3.2479675 | 2.4718101 | 150 | 492 | 337 |
| GBScompat_{—} | 2.3374384 | 3.5473934 | 1.8344371 | 406 | 422 | 151 |
| common_137 | | | | | | |
| GBScompat_{—} | 3.825 | 2.1508772 | 2.7226721 | 200 | 285 | 494 |
| common_132 | | | | | | |
| common_3378 | 3.1528302 | 2.4625407 | 2.084507 | 530 | 307 | 142 |
| common_710 | 3.1576642 | 1.7142857 | 1.9642857 | 685 | 70 | 224 |
| common_708 | 1.6122449 | 1.9786477 | 3.3483333 | 98 | 281 | 600 |
| common_1691 | 2.8 | 2.8907363 | 2.3759399 | 425 | 421 | 133 |
| common_766 | 2.604336 | 2.025 | 3.1 | 369 | 120 | 490 |

TABLE 3
Detailed information of each of the SNPs associated with botrytis resistance in Cannabis
as provided in Table 2. The “ref” reference allele based on the CS10 genome and the
identified “alt” alternative allele based on the SNP marker panel are given for each
SNP. The “context sequence” is given with the SNP given in brackets. All of the
sequences and alleles are provided with reference to the plus strand.

| SNP Name | ref | alt | Context sequence |
|---|---|---|---|
| common_3407 | C | A | GAAGGTCTAAAATCTTGAAAATGCTGCGACTTATATAAAGGAAAAT |
| | | | AAAATGAATGTTTAAATTTAGTTTCAATTTCAATTTCAATTTATAGTC |
| | | | TTAAGCAGGAAGCAGGGCCCTTCATCTTCCAGAAATTCAATGGAG |
| | | | AAGTAGTGAGACTCAGTGTGAGAGCGAGAGATGGAAGCCGTTGC |
| | | | AGAGGGGCTTTGGGGACT[C/A]GCGGACTACCACGAGAAGAAAG |
| | | | GAGACGTAGGGAAAGCTGTGAAGTGTCTCGAAGCCATTTGCCAG |
| | | | AGTCAGGTCACCTTCTTCCCCATCGTCGAGGTCAAGACTCGTCTT |
| | | | CGAATCGCTGCTTTACTTCTCAAGCATTCTCATAATGTTAATCACG |
| | | | CTAAGTCTCACCTCGAACGATCTCAGTTGCTTCTCAAGAACATC |
| | | | (SEQ ID NO: 5) |
| common_703 | G | A | ATTTATCTGCAAAAGATATAATGCACAAAAACAAACCTGTTCAGCA |
| | | | ATGCTAGCTGCTTCAGCATATGCATCTTGAATATTCTCAGCAAGCC |
| | | | TGTGGAGAAATATGTTTGATATTCCTCCGGCCGCAACAATAAATG |
| | | | GACCAGTGGCCAATGTTATGAGTGCAATCTGCCAACAGTTGACAA |
| | | | ATCCAATAACAAGACCAGC[G/A]AAAAATGTAGCCATGTTGTGAAC |
| | | | ATAATTTCCAACCTGTAATCAAAGAGGAACACAGACTTTAGACAAG |
| | | | AAATTTGAATCAACAAATCATATAGAAAGCTGAAATTTTTCATAACA |
| | | | AATAAAAATTCCCAAAAGGGAACATGAGCTAGCTAATATAAATTAG |
| | | | CATAAAATTACCTTTTCACTTAAAGCGGACTGAATGAGC |
| | | | (SEQ ID NO: 6) |
| common_719 | A | T | TCCTTTCTTTTTGGAGATTCAACTCTAATGACATTTTAAGTAGAGTT |
| | | | TTCTTCTTGGTTAATGGTACTAGGATTAAATTATTGCTATGTACAAT |
| | | | GGTAGAACTAGAAGCCAAGTTACTAACACAAAAGCTTAGTTCTTTT |
| | | | GAGAAGTGCAGGTGGAACTAATCAGCAGTGAGTCAGGAGCCATG |
| | | | GTTGAAGTTAAACACAT[A/T]GTGACACTTAAAAAGGATGAATTAAG |
| | | | TTCAGAAGTTGTGATATCAAACTCTAGCTCTGCTCCAGTTCAATTG |
| | | | ACTGGGTGTGTTCTGAGCCATCTGAGGGTGAGCTCTCCTGATGCA |
| | | | ACTTATGCAATCGGATTAGAAGGATCGGATTTCTTCTCCAGGCCT |
| | | | CCATTTTCATCAAACTTTGGTATTGTTCCTCCAGACTTT |
| | | | (SEQ ID NO: 7) |
| common_3408 | G | T | AGAAATAAAGATGGCGGTGATTGGTTGAAAGGAAAAACAGTAGCA |
| | | | GAAGCAAGTGCCTGCAGTGAGAAAGTGGGAAATTTTACCTGGGAA |
| | | | AGTGTAGCTGATCGGCGAGAGAATAGTATACAAATGCCATTCATA |
| | | | CTGGATGATAATATTGCAGCGAACAGAAACAAAGATAACAGTGAT |
| | | | TGGTTGAAAGGAAAAGCAGTA[G/T]CAGAAACAAGTGCCTTCAGTG |
| | | | AGAAAGTTGGAAATTTTTCTTGGGAAAATGTAGCTGATCGACCAG |
| | | | AGAAAAGTATTCAAGTATTTTTCAATGGAAAGATTGAAAAATTTCC |
| | | | CTGGAAAAATGTAGTTGGCCAACGGGAAAATACTAGTCAAATCCC |
| | | | ATTTATTAACGATGCAGCCAATGGAGAGAATGATGACAGTTCTT |
| | | | (SEQ ID NO: 8) |
| GBScompat_ | G | A | TGCAGCTCACTGCCTTAAACCGCATCTTTTCAACATATAGATCTGC |
| common_137 | | | AGAAATTTTAATACTGTGTTATATAATATGATCATTCTTTTAAAAAAA |
| | | | AAATCATTAAAATGGAGATAGCAACTTGGCTAAAACTCACCCATTA |
| | | | CACAAGTGTTCAAATTTGGAGCTGTTTTAAAGAGCCTGAAGAACAT |
| | | | AACATAGTTTCCAGA[G/A]GTAACAGCAGCACGAACTGCAAGAGCA |
| | | | TGCTTTACAGCATTATCCCTTTTTGCTTCCCTTGATAATCTGAACTC |
| | | | AATTTCACAACAATATTGAAGGTCATTACAAGAGTTTCAACATAGT |
| | | | AGAAGGATGGTAAATAGCTAATGAAATTTCCTCAAATCATAACTGA |
| | | | CCTTGACATGGATGATACAAGATCTCTGTTGTTAC |
| | | | (SEQ ID NO:9) |
| GBScompat_ | G | C | GCTACATTGTTTTGGTGTACCTAATGGATAACATTAGTTGGGAATT |
| common_132 | | | AGGGTTTGGGATTTGTCTTGCCATGACAGCACTTGGACTAATTTC |
| | | | GTTTTTGCTTGGAAAGAATTTCTATCGATTCGTTAAGCCTAAGGGA |
| | | | AGCCCATTTGTGAGCATGGCTAGAGTTGTCGTTGCAGCTATTCGG |
| | | | AATAGAAAATTGTCACTAA[G/C]TGATCGTGCTTTAACAAATACATA |
| | | | TACATCGGGTATCATTGTTCACACAAAATCACCAAGTAAACGTTTC |
| | | | AGGTATATTATATATAAAATATCCAACTAAATTATATATTGTAGAAA |
| | | | TAAATAATAGTAATGATTAACTCCCTTAAAATTTCAAAGTAGTATTA |
| | | | AGGGAAAAAGAAGGTCCTCAAATTTACTGTTGAATTG |
| | | | (SEQ ID NO: 10) |
| common_3378 | G | A | CTAGATTTCTTTGTCTTGAAATTTATTTTTCTTTTTGATCTGTTTCTT |
| | | | CTGGTTCTGATCTGTAAGGATCCGTGTATTTCACTCTTGGGTTTTT |
| | | | TTTTTTTTTTATCAGTTTAGGGGATTTGAGTTGCGATTTTTGTGTCG |
| | | | TTTGACTGTTTTGGTTTGCGATCTGGTGCTGATTTGTGTCGTTTGT |
| | | | GCGTTTTTGTAGCT[G/A]CGTCTGTGTAATAATGGCGTCGAAACGG |
| | | | ATCTTGAAGGAGCTCAAGGATTTGCAGAAAGATCCTCCTACCTCT |
| | | | TGCAGCGCCGGTATATTTCGTTACCTTAGTATTGTTCTCTTTTATAT |
| | | | GCGTAGCTTATATTGTTAATCCGGTGAACGGAAAGCTTTATCTTTC |
| | | | TGTGTGTTTATATAATATATTATCTATTTATTTAT |
| | | | (SEQ ID NO: 11) |
| common_710 | C | T | CCAAAATGAGCACATGAAAAAATAAAGTAGAATTAAGGAAAATGTT |
| | | | CTATACCCAAATTAATACATAGTCGAATGGTTAATTGGACTATATTA |
| | | | TAAAATTTAATTATTTATTTATAGGATTAATTGTTTGAATAAATATAT |
| | | | ATATACCTGCAAGAACTTGATGATATTGCTAGCGCCAAAGACTCTA |
| | | | TGAGCAATGGTGAA[C/T]TTAATAGGTTCGGTTGGCGGGAAATACG |
| | | | GCGCTAAAACGCACTTGTCGACACATCGGCGGCGAAGGATTTTG |
| | | | CAGGCAGCGCAGGGGCTCATGACGATAGCTGGTGGTGGAGGCG |
| | | | GTGGCGGAGGAGGAGTTGGACTAACATTATTATTACTAGTACTGG |
| | | | TGGGAGAAGATTGAGAGGAGGGTGGAGAAGATAACTGAGAT |
| | | | (SEQ ID NO: 12) |
| common_708 | G | A | TTGTAGAGGATCTGTGAAGGGGCTTTCTGTTTTACTTGAGCCTGT |
| | | | ATTTTTGTCACAACGGGATGTGCAAGAACTCTTGTGGATCAGCTTT |
| | | | CTCGCCAGCTCTATCCATTACCCTTCTCATCATTGATCTTATTGAA |
| | | | TCTGGTTGATGAAAATGGTCCAGATGAATAATAAAGCTCCCGTAA |
| | | | CCAATGATGAGCTAACTGA[G/A]CTGAATCACCGGATTCAAGATGG |
| | | | GAAAAAAGAAAGAAGGGAGAGGGTTACGAGAGAACGCCAAGGGC |
| | | | TCTCAGAGGAACATGAATCTAGGATCAATGAAGATGTGCAACAAC |
| | | | ATGTCAGCAATGGGGAGATTGGAACAGTACAGGCTCTGGAGAGG |
| | | | AGTCATTCTGGCCAGCGGAGGTCTCAGCAACAGCCTCAAGGACA |
| | | | T |
| | | | (SEQ ID NO: 13) |
| common_1691 | A | G | TAATATATTGAAATTGATTTTGTGCATTTAACAATTTTCATGTCATTT |
| | | | TGTGCACCCCTCTCCTATAATTTTTAATTATTATTTATATGTTTTGTT |
| | | | GATTAGGAAATATGAAGTTTAATTTTATGTCTATGTTTCAGATACAA |
| | | | TGGCGGTTCCCCTACTCACCAAGAAGATTGTGAAGAAGCGCGTCA |
| | | | AGCAGTTCAAGAA[A/G]CCCCAATGGGACAGGAAGATATCCGTCA |
| | | | AGGTTTGTAGTTCACCAATTATTCCTCATTTGTAGGTTTTATTATGA |
| | | | CTTAATCAGTATGCAAGTATTTGTAGGTACAAGTGTTTATCTTCTC |
| | | | ATCTATTTTGTAAAGACAATGTTGATAGCTTTGTTTATTTTAACATT |
| | | | TTCCTGTTCTGGTTTTCCCTTTTGATTTTAGTT |
| | | | (SEQ ID NO: 14) |
| common_766 | T | C | AACAATGAGGAGTTGTTGAATAACGAATTCTACATTGGACTTAGGC |
| | | | AAAGGAGAGCAACTGGGAAGGTCTAGTAACTTCTTGTTACATTTTA |
| | | | TTGATGTGTAAGGTTATAGTTTCCACTTTCAAAATGCAGTTAAATC |
| | | | AGCCTTGAGAATCTGTTTGCATTATGTACAGGAATATTCTGAGCTT |
| | | | TTGCACGAGTTCATGAC[T/C]GCAGTTAAGCAAAACTATGGGGAAA |
| | | | AAGTTCTTGTGCAGGTAAACATTTGATTTTAAAGCTACATTTTCAG |
| | | | CATATGCATGGATTTACTTGAGGTTTTCAGCATTCTATTTCTGTTG |
| | | | ATGTTTGCAGTTCGAAGATTTTGCAAACCACAATGCTTTTGATCTG |
| | | | CTAGCGAAATATGGAACCACGCATCTTGTCTTCAACGA |
| | | | (SEQ ID NO: 15) |

TABLE 4
Targeted sequencing primers (5′ to 3′) for the SNPs identified in Table 2, as described
in Example 1.

| | Forward | Reverse | Forward | Reverse |
|---|---|---|---|---|
| SNP Name | Primer 1 | Primer 1 | Primer 2 | Primer 2 |
| common_3407 | GAAGCAGGGCC | AGCAACTGAGAT | AAGCAGGGCCC | AGCAACTGAGAT |
| | CTTCATCTT | CGTTCGAGG | TTCATCTTC | CGTTCGAGG |
| | (SEQ ID NO: 16) | (SEQ ID NO: 17) | (SEQ ID NO: 18) | (SEQ ID NO: 17) |
| common_703 | TTTGATATTCCT | GCTCATTCAGTC | TTCTCAGCAAGC | GCTAGCTCATGT |
| | CCGGCCGC | CGCTTTAAGT | CTGTGGAG (SEQ | TCCCTTTTGG |
| | (SEQ ID NO: 19) | (SEQ ID NO: 20) | ID NO: 21) | (SEQ ID NO: 22) |
| common_719 | TGAGAAGTGCA | AGGCCTGGAGA | TGAGAAGTGCA | GGCCTGGAGAA |
| | GGTGGAACT | AGAAATCCG | GGTGGAACT | GAAATCCGA |
| | (SEQ ID NO: 23) | (SEQ ID NO: 24) | (SEQ ID NO: 23) | (SEQ ID NO: 25) |
| common_3408 | AAGTGTAGCTGA | TCTCCATTGGCT | GTGTAGCTGATC | TCTCCATTGGCT |
| | TCGGCGAG | GCATCGTT | GGCGAGAG | GCATCGTT |
| | (SEQ ID NO: 26) | (SEQ ID NO: 27) | (SEQ ID NO: 28) | (SEQ ID NO: 27) |
| GBScompat_ | CAGCTCACTGCC | TGCTGTAAAGCA | CAGCTCACTGCC | TCGTGCTGCTGT |
| common_137 | TTAAACCG | TGCTCTTGC | TTAAACCG | TACCTCTG |
| | (SEQ ID NO: 29) | (SEQ ID NO: 30) | (SEQ ID NO: 29) | (SEQ ID NO: 31) |
| GBScompat_ | TCGTTAAGCCTA | TTTGAGGACCTT | GGGAAGCCCAT | TTTGAGGACCTT |
| common_132 | AGGGAAGCC | CTTTTTCCCT | TTGTGAGCA | CTTTTTCCCT |
| | (SEQ ID NO: 32) | (SEQ ID NO: 33) | (SEQ ID NO: 34) | (SEQ ID NO: 33) |
| common_3378 | TCCGTGTATTTC | AAGATAAAGCTT | TCCGTGTATTTC | GATAAAGCTTTC |
| | ACTCTTGGGT | TCCGTTCACCG | ACTCTTGGGT | CGTTCACCG |
| | (SEQ ID NO: 35) | (SEQ ID NO: 36) | (SEQ ID NO: 35) | (SEQ ID NO: 37) |
| common_710 | TTGCTAGCGCCA | TCTTCTCCACCC | TTGCTAGCGCCA | TCTTCTCCACCC |
| | AAGACTCT | TCCTCTCA | AAGACTCT | TCCTCTCAA |
| | (SEQ ID NO: 38) | (SEQ ID NO: 39) | (SEQ ID NO: 38) | (SEQ ID NO: 40) |
| common_708 | TGGATCAGCTTT | GCTGGCCAGAA | TGTGGATCAGCT | GCTGGCCAGAA |
| | CTCGCCAG | TGACTCCTC | TTCTCGCC | TGACTCCTC |
| | (SEQ ID NO: 41) | (SEQ ID NO: 42) | (SEQ ID NO: 43) | (SEQ ID NO: 42) |
| common_1691 | AGATACAATGGC | TCAAAAGGGAAA | GATACAATGGCG | TCAAAAGGGAAA |
| | GGTTCCCC | ACCAGAACAGG | GTTCCCCT | ACCAGAACAGG |
| | (SEQ ID NO: 44) | (SEQ ID NO: 45) | (SEQ ID NO: 46) | (SEQ ID NO: 45) |
| common_766 | GGAGAGCAACT | GGTTTGCAAAAT | AGGCAAAGGAG | TCGAACTGCAAA |
| | GGGAAGGTC | CTTCGAACTGC | AGCAACTGG | CATCAACAGA |
| | (SEQ ID NO: 47) | (SEQ ID NO: 48) | (SEQ ID NO: 49) | (SEQ ID NO: 50) |

Used individually, the associated SNPs identified that define the three QTLs for Botrytis resistance can distinguish Botrytis resistant plants or populations of cannabis from less resistant plants or populations. The inventors propose that QTL1, QTL2, and QTL3 also act in combination to contribute to Botrytis resistance. To test this the inventors evaluated the most significant markers from each of the three QTLs (common-703, common_1691, common-340), in combination, in 978 genotyped plants from the populations used in the above GWA, for Botrytis resistance (Table 2). Similarly, a scale from 1-9 was used, where 1 indicated highly resistant plants and 0 indicated plants that are not resistant. The inventors found that in plants where common_703 is AA, common_1691 is GG, and common_3407 is CC on average these plants were more susceptible to Botrytis than in any other allelic state tested (Table 5). This indicates that the QTLs identified contribute to Botrytis resistance in combination and that the markers are effective in combination to identify and predict plants that are susceptible to Botrytis. The use of these markers alone and in combination, conducted by someone skilled in the art, is effective at improving the selection of plants that are susceptible to Botrytis in order to purge them from the breeding population improving the likelihood that the breeding population will have improved resistance to Botrytis. By contrast plants are highly resistant where SNP common_703 is GG, common_1691 is AA, and common_3407 is AA (Table 5). Several combinations of allele state conferred broad resistance. Each QTL alone contributed to some Botrytis resistance; however, the average resistance was improved when more than one of the markers for each QTL were in allelic states are predicted to contribute to resistance. When all three markers representing allele states that were predicted to contribute to resistance, the average resistance was improved the most.

TABLE 5
Combinatorial effect of QTL 1, 2, and 3 on Botrytis resistance. The three
most significantly associated SNPs common_703, common_1691, and
common_3407 for each QTL1, QTL2, and QTL3, respectively, are evaluated
for their effect in combination on average to the resistance of plants with
those alleles to Botrytis. Mean indicates the average resistance scored from
1-9 where 1 is most resistant. Count indicates the number of plants and frequency
is the occurrence of that particular allele combination in all plants tested.

| common_703 | common_1691 | common_3407 | Mean | Count | Frequency (%) |
|---|---|---|---|---|---|
| AA | AA | AA | 2.25 | 12 | 1.226993865 |
| AA | AG | AA | 2.538461538 | 39 | 3.987730061 |
| AA | GG | AA | 3.026315789 | 38 | 3.885480573 |
| AG | AA | AA | 1.166666667 | 6 | 0.613496933 |
| AG | AG | AA | 1.529411765 | 17 | 1.738241309 |
| AG | GG | AA | 2.245283019 | 53 | 5.419222904 |
| GG | AA | AA | 1.5 | 8 | 0.81799591 |
| GG | AG | AA | 1.666666667 | 27 | 2.760736196 |
| GG | GG | AA | 1.666666667 | 27 | 2.760736196 |
| AA | AA | AC | 2.555555556 | 27 | 2.760736196 |
| AA | AG | AC | 3.208791209 | 91 | 9.304703476 |
| AA | GG | AC | 3.818181818 | 66 | 6.748466258 |
| AG | AA | AC | 1.5 | 20 | 2.044989775 |
| AG | AG | AC | 2.240740741 | 54 | 5.521472393 |
| AG | GG | AC | 2.382022472 | 89 | 9.100204499 |
| GG | AA | AC | 2 | 16 | 1.63599182 |
| GG | AG | AC | 1.68627451 | 51 | 5.214723926 |
| GG | GG | AC | 1.813953488 | 43 | 4.396728016 |
| AA | AA | CC | 3.5625 | 32 | 3.27198364 |
| AA | AG | CC | 3.878504673 | 107 | 10.9406953 |
| AA | GG | CC | 4.547169811 | 53 | 5.419222904 |
| AG | AA | CC | 2.3 | 10 | 1.022494888 |
| AG | AG | CC | 3.043478261 | 23 | 2.351738241 |
| AG | GG | CC | 3.181818182 | 33 | 3.374233129 |
| GG | AA | CC | 1 | 2 | 0.204498978 |
| GG | AG | CC | 2.333333333 | 15 | 1.533742331 |
| GG | GG | CC | 2.631578947 | 19 | 1.942740286 |

The inventors reasoned that the QTLs identified could contribute to broader resistance to pathogens beyond Botrytis. The F2 populations that were used in the experiment for Botrytis were also scored for general resistance at the same time they were scored for Botrytis resistance. Cannabis F2 plants deriving from different populations were harvested at maturity between October and November 2021. Plants were phenotypically assessed for general resistance using a subjective scale from 1 to 9. In this scale, “1” indicates plants with low resistance while a “9” indicates plants with high resistance.

As above, the inventors evaluated the most significant markers, common_703, common_1691, common_3407, from each of the three QTLs in the 978 plants that were genotyped previously for use in the GWA in combination for general resistance, Table 2. Similarly for Botrytis resistance we found that the QTLs identified contribute to general resistance as phenotyped. Table 6 shows results that indicate that the allele state of the SNPs that are predictive of Botrytis resistance are also predictive of general resistance and that in combination they show a greater affect. For example, plants with the allelic variants common_703 GG, common_1691 AA, common_3407 AA are resistant to Botrytis, this was also found to be true for general resistance (Table 6). Further supporting this, the allelic variants that are associated with loss of Botrytis resistance in these three QTLs are also indicative of a loss of general resistance.

TABLE 6
Combinatorial effect of QTL 1, 2, and 3 on general resistance. The three most
significantly associated SNPs common_703, common_1691, and common_3407
for each QTL1, QTL2, and QTL3, respectively, are evaluated for their effect
in combination on average to the general resistance of plants with those alleles.
Mean indicates the average general resistance scored from 1-9 where 9 is most
resistant. Count indicates the number of plants and frequency is the occurrence
of that particular allele combination in all plants tested.

| common_703 | common_1691 | common_3407 | Mean | Count | Frequency (%) |
|---|---|---|---|---|---|
| AA | AA | AA | 5.666666667 | 12 | 1.226993865 |
| AA | AG | AA | 5.871794872 | 39 | 3.987730061 |
| AA | GG | AA | 4.342105263 | 38 | 3.885480573 |
| AG | AA | AA | 7.666666667 | 6 | 0.613496933 |
| AG | AG | AA | 7 | 17 | 1.738241309 |
| AG | GG | AA | 5.339622642 | 53 | 5.419222904 |
| GG | AA | AA | 7 | 8 | 0.81799591 |
| GG | AG | AA | 7.074074074 | 27 | 2.760736196 |
| GG | GG | AA | 6 | 27 | 2.760736196 |
| AA | AA | AC | 5.740740741 | 27 | 2.760736196 |
| AA | AG | AC | 5.527472527 | 91 | 9.304703476 |
| AA | GG | AC | 4.636363636 | 66 | 6.748466258 |
| AG | AA | AC | 7.3 | 20 | 2.044989775 |
| AG | AG | AC | 6.222222222 | 54 | 5.521472393 |
| AG | GG | AC | 6.112359551 | 89 | 9.100204499 |
| GG | AA | AC | 6.75 | 16 | 1.63599182 |
| GG | AG | AC | 6.921568627 | 51 | 5.214723926 |
| GG | GG | AC | 6.581395349 | 43 | 4.396728016 |
| AA | AA | CC | 5.09375 | 32 | 3.27198364 |
| AA | AG | CC | 5.056074766 | 107 | 10.9406953 |
| AA | GG | CC | 4.698113208 | 53 | 5.419222904 |
| AG | AA | CC | 6.3 | 10 | 1.022494888 |
| AG | AG | CC | 5.956521739 | 23 | 2.351738241 |
| AG | GG | CC | 5.606060606 | 33 | 3.374233129 |
| GG | AA | CC | 7.5 | 2 | 0.204498978 |
| GG | AG | CC | 6.6 | 15 | 1.533742331 |
| GG | GG | CC | 5.947368421 | 19 | 1.942740286 |

### Example 2

### Gene Identification

There are presently no known genes identified in Cannabis that have been shown to regulate Botrytis resistance or general resistance in Cannabis. The genetic regulation of disease resistance has been described and characterized in several plant species, however the multitude of different genes involved in this process does not easily allow the identification of disease resistance genes in Cannabis. The inventors considered genes that may influence disease resistance related to basal resistance, signal transduction, genes that may be related to reactive oxygen species, those that have homologs in other organisms that contribute to disease resistance. They next sought to identify putative genes that could encode proteins that may be responsible for changes in Botrytis or general resistance based on SNPs present that could alter that protein's function or expression. Using the findings of the association studies they identified candidate genes at the QTL identified.

The inventors determined that SNP differences between cannabis genomes could inform which genes play a role in the trait of interest. Short reads from sequenced lines were dereplicated with NGSReadsTreatment (version 1.3, Gaia et al., 2019) and pre-processed with fastp (version 0.23.2, Chen et al., 2018). Reads were aligned to the CS10 reference genome with Bowtie2 (version 2.3.5.1, with options --rg and --rg-id to add read-group identifiers, Langmead and Salzberg, 2012). Only unique alignments with a mapping quality of at least 10 were kept. SNPs were called with freebayes and filtered for a minimal quality of 20 (version v1.3.2-40-gcce27fc, parameters -p 2 --min-coverage 20 -g 30000 --min-alternate-count 4 --min-alternate-fraction 0.1 --min-mapping-quality 10 --max-complex-gap -1, Garrison and Marth, 2012). SNPs were finally filtered for a coverage between 5 and 10,000 within each line and annotated with Ensembl Variant Effect Predictor (version 106.1, McLaren et al., 2016).

For each line, the inventors constructed a pseudogenome by incorporating its variants into the CS10 reference genome with vcf-consensus (Danecek et al., 2011). CS10 annotation was lifted over, to align genes from a reference genome to a target genome, with liftoff (version 1.6.3, Shumate and Salzberg, 2021). Protein and cDNA sequences were extracted with custom scripts. Proteins and cDNA sequences for a given protein/transcript from all lines were aligned with muscle (v3.8.31, Edgar, 2004).

Proteins from each of the three QTLs, QTL1: NC_044371.1 being located between 20201111-24661598 bp, QTL2: NC_044373.1 being located between 70000-90000 bp, QTL3: NC_044375.1 being located between 85855033-91075725 bp were extracted.

Multiple alignments from protein sequences were converted to tables including the variant positions and protein variants were tested for correlation with the significant SNPs from the GWAS marker panel. Only proteins with significant associations were kept. These proteins were then used to extract all SNPs with the associated genes. SNPs were also tested for association with the significant SNPs from the GWAS marker panel. Only significant SNPs with an effect on the coding sequence were kept and associated proteins with homologs in Arabidopsis were used as candidates (Table 7).

The polymorphisms at the given position in the gene candidates in Table 7 in each of the three QTL, QTL1-3, are considered to represent a haplotype that contributes to variance in Botrytis resistance. Given the complexity of fungal resistance response in plants, multiple contributors to resistance is not unusual and contributes to the difficulty in identifying specific genetic determinants of resistance. The inventors inspected the gene candidates for effect of the polymorphism likelihood to impact protein function, the predicted function of the respective encoded protein, and homology to known proteins in other plants that have been implicated in fungal resistance.

The investigators determined that at QTL1 the Protein kinase domain-containing protein (PKD) (NCBI accession: XP_030492442.1) contributes to Botrytis resistance. A SNP at position 23309645 on chromosome NC_044371.1 leads to a frameshift mutation disrupting the function of the resultant protein. The reference sequence for this SNP can be found in Table 8. The loss of function of this protein kinase domain containing protein will result in the variance in Botrytis by as yet discovered mechanism. Disruption in the perception of fungal elicitors of the plant immune response is likely. Alternatively, Botrytis may subvert host defence through the activation of this receptor pathway. Its disruption may nullify this.

The investigators also determined that at QTL3, PTI6-like protein (NCBI accession: XP_030505675.1) contributes to Botrytis resistance. A SNP at position 86872263 (C/A) on chromosome NC_044375.1 leads to a missense variant Proline (P) 111 to Threonine (T) 111, with respect to CS10. PTI6-like is 51% identical to the PTI6 in Solanum lycopersicum. The reference sequence for this SNP can be found in Table 8. The proline at amino acid position 110 is conserved in Solanum as well as in many other plants species with PTI6 homologs, indicating it may be critical to the protein's function. A SNP that leads to the loss of proline at this position or a change to proline likely impacts the secondary structure of the protein. Interestingly P110 is situated close to the proposed DNA binding domain of this protein, suggestion that a change in structure could impact its DNA binding activity. PTI6 in Solanum has been characterized as being involved in the basal plant defence response. Overexpression of PTI6 in Arabidopsis and Solanum can improve plant defence responses to bacterial challenge. PTI6-like is a AP2/ERF family transcription factor, this class of protein has been shown to bind to the GCC-box pathogenesis-related promoter.

TABLE 7
A list of genes derived from the three identified QTLs on NC_044371.1, NC_044373.1, and NC_044375.1. The chromosome,
position of the SNP variant, the false discovery rate (FDR) for the SNP variant, the type of variant, the predicted Severity
of the Variant on the protein, the protein ID and its description with reference to the CS10 reference genome is given.

| | | | Severity of | Type of | | Protein |
|---|---|---|---|---|---|---|
| Chromosome | Position | FDR | Variant | Variant | Protein ID | description |
| NC_044371.1 | 21138917 | 9.65E−07 | MODERATE | missense variant | XP_030486980.1 | Phosphatidylinositol/ |
| | | | | | | phosphatidylcholine |
| | | | | | | transfer protein SFH3 |
| NC_044371.1 | 22992456 | 1.22E−07 | MODERATE | missense variant | XP_030486999.1 | NA |
| NC_044371.1 | 19129320 | 0.00035193 | MODERATE | missense variant | XP_030487105.1 | Transcription factor protein |
| NC_044371.1 | 21152562 | 7.52E−05 | HIGH | frameshift variant | XP_030490359.1 | none |
| NC_044371.1 | 21152562 | 7.52E−05 | HIGH | frameshift variant | XP_030490360.1 | NA |
| NC_044371.1 | 23381911 | 5.98E−07 | MODERATE | missense variant | XP_030490446.1 | Putative white-brown complex protein |
| NC_044371.1 | 19144317 | 5.60E−10 | MODERATE | missense variant | XP_030490663.1 | Protein UPSTREAM OF FLC |
| NC_044371.1 | 22539044 | 0.00085391 | MODERATE | missense variant | XP_030491140.1 | Cysteine proteinase inhibitor |
| NC_044371.1 | 23296425 | 3.11E−06 | MODERATE | inframe insertion | XP_030491256.1 | 3-5 exonuclease domain-containing |
| | | | | | | protein |
| NC_044371.1 | 19414742 | 2.14E−07 | MODERATE | inframe insertion | XP_030491968.1 | Ankyrin repeat-containing protein |
| NC_044371.1 | 23558239 | 0.00061318 | MODERATE | missense variant | XP_030492275.1 | Pentatricopeptide repeat-containing |
| | | | | | | protein |
| NC_044371.1 | 19175774 | 2.32E−12 | LOW | synonymous variant | XP_030487289.1 | Receptor-like protein |
| | | | | | | kinase THESEUS 1 |
| NC_044371.1 | 19177444 | 8.57E−09 | LOW | synonymous variant | XP_030487290.1 | F-box domain-containing protein |
| NC_044371.1 | 23603321 | 4.62E−06 | LOW | synonymous variant | XP_030489715.1 | Receptor-like protein kinase HERK 1 |
| NC_044373.1 | 766230 | 0.00054453 | MODERATE | missense variant | XP_030496175.1 | none |
| NC_044373.1 | 690221 | 1.39E−06 | MODERATE | missense variant | XP_030496462.1 | Acyl-[acyl-carrier-protein] hydrolase |
| NC_044373.1 | 667147 | 0.00058258 | MODERATE | missense variant | XP_030496617.1 | Sec-independent protein translocase |
| | | | | | | protein TATC, chloroplastic |
| NC_044373.1 | 890383 | 1.28E−06 | MODERATE | missense variant | XP_030499379.1 | NA |
| NC_044375.1 | 89181826 | 8.87E−08 | MODERATE | missense variant | XP_030503049.1 | Putative transcription |
| | | | | | | factor Hap3/NF-YB family |
| NC_044375.1 | 89755507 | 2.16E−08 | MODERATE | missense variant | XP_030503058.1 | Pentatricopeptide repeat- |
| | | | | | | containing protein |
| NC_044375.1 | 89293166 | 6.24E−08 | MODERATE | missense variant | XP_030503091.1 | Serine/threonine-protein kinase haspin |
| NC_044375.1 | 85277440 | 2.72E−07 | MODERATE | missense variant | XP_030503125.1 | ABC transporter G family member 31 |
| NC_044375.1 | 89254353 | 1.42E−08 | MODERATE | missense variant | XP_030503274.1 | Pleiotropic drug resistance protein 3 |
| NC_044375.1 | 89112636 | 2.72E−08 | MODERATE | inframe deletion | XP_030503299.1 | FACT complex subunit SPT16 |
| NC_044375.1 | 86995086 | 6.10E−05 | MODERATE | missense variant | XP_030503352.1 | Methylesterase 17 |
| NC_044375.1 | 86933716 | 2.22E−06 | MODERATE | missense variant | XP_030503487.1 | Methylesterase 17 |
| NC_044375.1 | 85974683 | 3.28E−08 | LOW | stop retained variant | XP_030503668.1 | Internal alternative NAD(P)H-ubiquinone |
| | | | | | | oxidoreductase A1, mitochondrial |
| NC_044375.1 | 85782385 | 3.30E−06 | MODERATE | missense variant | XP_030503871.1 | Zinc finger CCCH domain-containing |
| | | | | | | protein 66 |
| NC_044375.1 | 88883710 | 2.43E−09 | LOW | splice | XP_030504082.1 | NA |
| | | | | polypyrimidine tract | | |
| | | | | variant; splice | | |
| | | | | region variant; | | |
| | | | | intron variant | | |
| NC_044375.1 | 89394419 | 2.43E−09 | MODERATE | missense | XP_030504167.1 | Aspartic proteinase |
| | | | | variant; splice | | |
| | | | | region variant | | |
| NC_044375.1 | 89394419 | 2.43E−09 | MODERATE | missense variant; splice | XP_030504168.1 | Aspartic proteinase |
| | | | | region variant | | |
| NC_044375.1 | 88537031 | 7.67E−06 | MODERATE | missense variant | XP_030504340.1 | transcription factor MYB48-like isoform |
| NC_044375.1 | 88537031 | 7.67E−06 | MODERATE | missense variant | XP_030504341.1 | NA |
| NC_044375.1 | 89924198 | 1.66E−06 | MODERATE | missense variant | XP_030504526.1 | Calcium uniporter protein |
| NC_044375.1 | 89359731 | 5.23E−11 | MODERATE | missense variant | XP_030504611.1 | Succinate dehydrogenase subunit 5, |
| | | | | | | mitochondrial |
| NC_044375.1 | 85285500 | 4.10E−07 | MODERATE | missense variant | XP_030505452.1 | NA |
| NC_044375.1 | 85297298 | 4.10E−07 | MODERATE | missense variant | XP_030505453.1 | NA |
| NC_044375.1 | 86009965 | 4.91E−07 | MODERATE | missense variant | XP 030505459.1 | CACTA transposable element |
| NC_044375.1 | 86830988 | 8.69E−08 | MODERATE | missense variant | XP_030505461.1 | NA |
| NC_044375.1 | 87544677 | 6.10E−05 | MODERATE | missense variant | XP_030505466.1 | Gag-protease polyprotein |
| NC_044375.1 | 89191384 | 2.43E−09 | MODERATE | missense variant | XP_030505479.1 | Peroxidase |
| NC_044375.1 | 89721736 | 6.31E−07 | HIGH | stop gained | XP_030505485.1 | NA |
| NC_044375.1 | 87437592 | 2.16E−08 | MODERATE | missense variant | XP_030505574.1 | Superoxide dismutase |
| NC_044375.1 | 86872263 | 2.16E−08 | MODERATE | missense variant | XP_030505675.1 | pathogenesis-related genes transcriptional |
| | | | | | | activator PTI6-like |
| NC_044375.1 | 85848327 | 4.56E−09 | MODERATE | missense variant | XP_030505737.1 | Microsomal delta 15 desaturase |
| NC_044375.1 | 85848327 | 4.56E−09 | MODERATE | missense variant | XP_030505738.1 | Microsomal delta 15 desaturase |
| NC_044375.1 | 89770991 | 1.66E−06 | MODERATE | missense variant | XP_030506212.1 | Germin-like protein subfamily 2 member 4 |
| NC_044375.1 | 88948018 | 2.43E−09 | MODERATE | missense variant | XP_030506308.1 | Pentatricopeptide repeat-containing |
| | | | | | | protein |
| NC_044375.1 | 88948018 | 2.43E−09 | MODERATE | missense variant | XP_030506309.1 | Pentatricopeptide repeat-containing |
| | | | | | | protein |
| NC_044375.1 | 86769160 | 2.16E−08 | LOW | synonymous variant | XP_030504156.1 | Putative LRR receptor-like |
| | | | | | | serine/threonine-protein kinase |
| NC_044375.1 | 86769160 | 2.16E−08 | LOW | synonymous variant | XP_030504157.1 | Putative LRR receptor-like |
| | | | | | | serine/threonine-protein kinase |

TABLE 8
Detailed information of the two polymorphisms determined to contribute to Botrytis
resistance in QTL1 and QTL3 (possible causative polymorphisms). The chromosome and
position are given based on the CS10 reference genome. The “context sequence” is
given with the polymorphism provided in square brackets. The first mentioned sequence
in square brackets is the reference allele based on the CS10 genome and the identified
alternative allele based on the polymorphism detected is the second mentioned sequence
in square brackets. All of the sequences and alleles are provided with reference to
the plus strand.

| Polymorphism | | | |
|---|---|---|---|
| Name | Chromosome | Position | Context sequence |
| QTL1_PKD | NC_044371.1 | 23309645 | TGTATCTTGGGTTCGACTACATGGGTACTCTTCT |
| | | | TAGACTTGCACATGAG[TACACACAAAT/TACACA |
| | | | CACAAATJACACACAAATTGATTAAGCACAAATTT |
| | | | TGATCTTTGTGATGGTGATTGTT |
| | | | (SEQ ID NO: 51 and SEQ ID NO: 52) |
| QTL3_PTI6-like | NC_044375.1 | 86872263 | AATCACTTATTACTAAGCAAGAAAGAGTCTCAAA |
| | | | GAGACCGATTGGATCA[C/A]CGGAATCGGATTCC |
| | | | GTTCGTCGGAAAAAGTTCAGAGGCGTTCGCCGA |
| | | | AGA (SEQ ID NO: 53) |

### Example 3

### Validation of QTL in Selection for Botrytis Resistance

The inventors conceived to evaluate the identified QTL and SNP markers in predicting plants with resistance to Botrytis for marker-based selection through the use of a training population of 234 diverse cannabis genotypes that included high THC varieties, low THC/high CBD varieties, and assorted hemp plants. The training population was grown and harvested in an open field in 2022. The inventors determined the genotypes and phenotypes of these plants.

To determine genotype, DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel was employed. The inventors designed the marker panel based on a pangenome comprising 13 genotypes in order to include SNP markers common to a broad diversity of HRT and hemp cannabis, including the Cannabis Sativa CS10 reference genome. Targeted sequencing was carried out on the Ion Torrent system by Thermo Fisher Scientific. The primers for the SNP identified are provided in Table 11. The library pool was loaded onto Ion 550 chips with Ion Chef and sequenced with Ion GeneStudio S5 Plus according to the manufacturer's instructions (Ion 550™ Kit from Thermo Fisher Scientific).

To determine phenotype, at the time of harvest whole plants including flower, leaf, and stem were scored for susceptibility to Botrytis cinerea. This trait has been quantified using a subjective scale from 0 to 5, where a score of 0 indicates plants with high resistance to Botrytis cinerea, while a score of 5 indicates plants with high susceptibility to Botrytis i.e., showing the presence of Botrytis disease symptoms and Botrytis conidiophore. Fungal pathogens that mimic the disease symptoms of Botrytis cinerea cannot be ruled out, however visual identification of Botrytis is commonplace.

The Botrytis resistance scores from 0-5, where 0 indicates most resistant plants, were used as an input for GWAS.

For consistency with the original model, the training population data underwent a round of imputation for reducing the number of missing values. In order to reduce missing data in the genotype file, an imputation has been performed using the HapMap_imputation software (GitHub-mwylerCH/HapMap_Imputation). Briefly, the genotype file is converted to a hapmap format (comma separated, http://augustogarcia.me/statgen-esalq/Hapmap-and-VCF-formats-and-its-integration-with-onemap/#hapmap).

In a first step, HapMap_Imputation counts the occurrence of each nucleotide at every single genotyped position. The most common nucleotide is defined as major allele, the second is defined as minor allele. Missing genotyping information is excluded. In the case major and minor alleles occur at the same number, the nucleotide of the reference cs10 genome is chosen as major allele. Subsequently, HapMap_Imputation sorts markers by position and parses the hapmap into the required fastPHASE (Scheet and Stephens, 2006) input format. Briefly, HapMap_Imputation splits the haplotypes into two separate rows, converts major and minor alleles into 0 and 1 respectively and produces temporary files for each chromosome.

During the third step, HapMap_Imputation downloads the latest fastPHASE version and runs the imputation using 8 cores in parallel. fastPHASE is run with ten random starts of the imputation algorithm. After imputation, HapMap_Imputation reverses the 0 and 1 coding into the major and minor nucleotide, respectively. Subsequently, the two haplotypes are combined, and the separate chromosomes are merged into a single file.

This combination of techniques resulted in 4461 SNP markers after filtering using 234 plants. The GWA was performed using GAPIT version 3 (Wang and Zhang, 2021) with a Mixed Linear Model (MLM). SNPs surpassing a LOD (−log₁₀(p-value)) value of 5 were considered to have a significant association with trait variation.

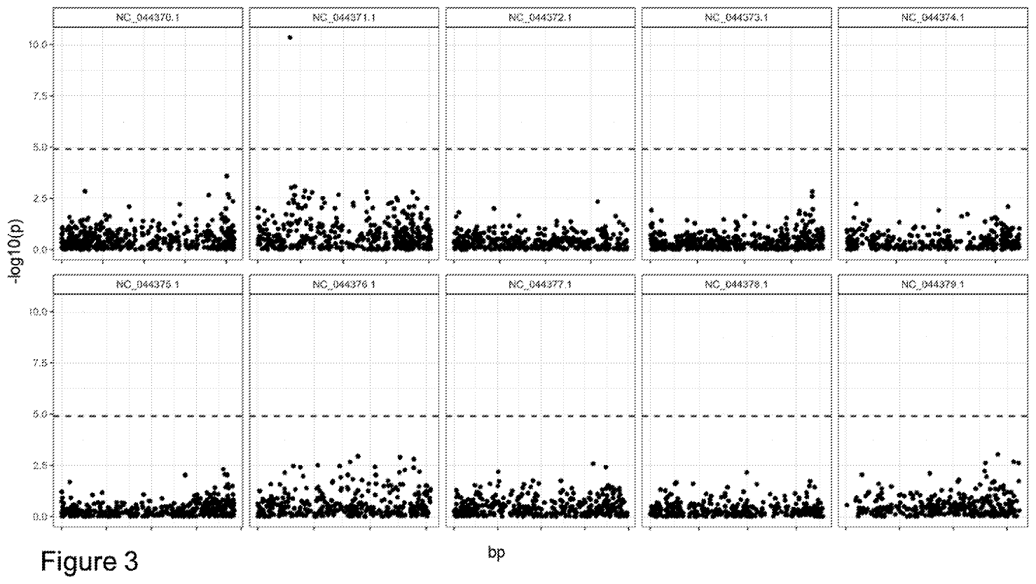

The GWA identified a single marker with a LOD score greater than 5 in the originally identified QTL1, “common_701” at position 18901130 on NC_044371 with respect to the CS10 genome (Table 9, FIG. 3). A reference or context sequence for the SNP “common_701” is provided in Table 10, while exemplary primers that can be used to detect the SNP are provided in Table 11. The identification of QTL1 by GWA in the training population is strong validation of this QTL and the use of markers based on this QTL to predict for Botrytis resistance. The GWA was not expected to pick up all originally identified QTLs as the number of plants in the training population were few and the plants used did not belong to a structured population as in the original experiment in Example 1. Additionally, the environmental and pathogen prevalence pressures in the 2022 field season were not identical to 2021, due to variables outside the inventors' control.

From the results of the GWA, the inventors validated the finding that QTL1 and the specific markers identified, in Example 1, Table 2, and including the SNP marker “common_701”, in the GWA are instructive in enabling someone skilled in the art to perform marker assisted selection and marker-based identification to select for plants resistant to Botrytis.

This is supported clearly when evaluating the allelic effect of the markers “common_703” (QTL1), “common_1691” (QTL2), and “common_3407” (QTL3) on average Botrytis resistance in the training population using a combination of these markers (Table 12). For reference, Table 12, evaluates the allele states of these three markers and identifies plants and the average allele effect, in combination, on Botrytis resistance. Marker “common_703” in allele state GG is clearly associated with resistance to Botrytis, as demonstrated in Example 1. Furthermore, regardless of the allele states of the other two markers it represents an excellent marker for the selection of resistant plants. Notably when selecting based plants based on all three markers in combination, where “common_703” is GG, “common_1691” is AA and “common_3407” is AA, the mean phenotype for Botrytis resistance of the plants with this combination of allele states is 0, indicating strong resistance to Botrytis. The combinatorial data also indicates that “common_1691” (QTL2) and “common_3407” (QTL3) are highly predictive for Botrytis resistance in the field trial in 2022. It can also be observed that when the alternative allele state of “common_703” is AA, “common_1691” is GG and “common_3407” is CC is determined, on average these plants are susceptible to Botrytis, this includes heterozygous states of the allele as shown in Table 12.

TABLE 9
GWA results of a SNP, “common_701” in QTL1 at position 18901130
on chromosome NC 044371 with reference to the CS10 genome, associated with
Botrytis resistance in the training population. The presence of the resistance
to Botrytis is predicted by the occurrence of the indicative allele (marked with *).
The LOD score for the mixed linear model is provided as LOD. “Mean_1” denotes
the average phenotypic value associated with Allele_1 based on scoring for resistance,
“Mean_2” denotes the average phenotypic value associated with Allele_2 based on
scoring for resistance and Mean_3 denotes the average phenotypic value associated with
Allele_3 based on scoring for resistance. Count_1, Count_2, and Count_3 denote
the number of plants that contributed to the average phenotypic value of
Mean_1, Mean_2, and Mean_3 respectively.

| SNP | LOD | Alleles_1 | Alleles_2 | Alleles_3 | Mean_1 | Mean_2 | Mean_3 | Count_1 | Count_2 | Count_3 |
|---|---|---|---|---|---|---|---|---|---|---|
| common_701 | 6.90529961 | CC* | CT | TT | 0.76262626 | 1.63636364 | 1.16666667 | 198 | 33 | 12 |

TABLE 10
Detailed information of the SNP associated with botrytis resistance in Cannabis as
provided in Table 9. The “ref” reference allele based on the CS10 genome and the
identified “alt” alternative allele based on the SNP marker panel are given for each
SNP. The “context sequence” is given with the SNP given in brackets. The sequence and
alleles are provided with reference to the plus strand.

| SNP | Ref | Alt | Context sequence |
|---|---|---|---|
| common_701 | C | T | TATAAAAACTCTCCTCCATATGATCGGCTCCTATACCATTACTATAA |
| | | | CTAACTAACTAACTAGCTATTTATATATATATATATATATATATTATAA |
| | | | TATAAAAATATTATATGTATGTGAATGTGATGAACATAATATAAATCA |
| | | | TATATATGTAACCAAAGAAACTGATTTCCCAAGCTGAAGAATCTTGA |
| | | | TATCATTTTC[C/T]AAAGGTGTGAGGTTCAAATCCAAACACAAAACCC |
| | | | TATTGTTCTTCTTATTCACCATCGCGGCCGGCCGCCGTACTTGCAA |
| | | | AGAACCGGCAATATCAACAGGCGGTGGCCGTGTGGCGGCCGAGG |
| | | | CCGCTGACAAAGCATGATGATGAGTCTGGTCGGTCAAGGCGGCTC |
| | | | TATGGCGCCTCATGTGGCCCCCCAAGGCTTGG |
| | | | (SEQ ID NO: 54) |

TABLE 11
Targeted sequencing primers (5′ to 3′) for the SNP identified in Table 9, as described
in Example 3.

| SNP | Forward Primer 1 | Reverse Primer 1 | Forward Primer 2 | Reverse Primer 2 |
|---|---|---|---|---|
| common_701 | TCCTCCATATGA | CCACCGCCTGTT | TCCTCCATATGA | GCCACCGCCTGT |
| | TCGGCTCCT | GATATTGC | TCGGCTCCT | TGATATTG |
| | (SEQ ID NO: 55) | (SEQ ID NO: 56) | (SEQ ID NO: 55) | (SEQ ID NO: 57) |

TABLE 12
Combinatorial allele states predictive of Botrytis resistance in the training population.
The three most significantly associated SNPs, common_703, common_1691, and
common_3407 for each QTL1, QTL2, and QTL3, respectively, are evaluated for their
effect in combination on average to the Botrytis resistance of plants with those
alleles based on scoring for resistance on a scale of 0-5, where 0 indicates most
resistant plants. The allele state that is predictive of Botryits resistance is shown
in parenthesis in the first row. The mean phenotype for Botrytis resistance in specific
plants with a combination of the three markers specific allele state is shown. The
number of plants with the combination of these markers is given as plant count and
the frequency of their appearance in the training population studied is noted.

| common_703 | common_1691 | common_3407 | | | |
|---|---|---|---|---|---|
| (GG) | (AA) | (AA) | Mean | Count | Frequency (%) |
| AA | AG | CC | 2.235294118 | 17 | 7.4235808 |
| AA | GG | CC | 1.5 | 2 | 0.8733624 |
| AA | AG | AC | 1.294117647 | 17 | 7.4235808 |
| AA | GG | AC | 1 | 1 | 0.4366812 |
| AA | AA | AA | 0.222222222 | 9 | 3.930131 |
| AA | AA | AC | 0 | 3 | 1.3100437 |
| AA | AA | CC | 0 | 2 | 0.8733624 |
| AA | AG | AA | 0 | 2 | 0.8733624 |
| AA | GG | AA | 0 | 1 | 0.4366812 |
| AG | AG | CC | 1.75 | 8 | 3.4934498 |
| AG | GG | AC | 1.6 | 5 | 2.1834061 |
| AG | AA | AC | 1.444444444 | 9 | 3.930131 |
| AG | AG | AC | 1.44 | 50 | 21.8340611 |
| AG | AG | AA | 0.5 | 6 | 2.6200873 |
| AG | AA | AA | 0.476190476 | 21 | 9.1703057 |
| AG | AA | CC | 0 | 2 | 0.8733624 |
| AG | GG | AA | 0 | 1 | 0.4366812 |
| AG | GG | CC | 0 | 1 | 0.4366812 |
| GG | AG | CC | 2 | 1 | 0.4366812 |
| GG | AA | CC | 1 | 3 | 1.3100437 |
| GG | AG | AC | 1 | 1 | 0.4366812 |
| GG | GG | AC | 0.666666667 | 3 | 1.3100437 |
| GG | AG | AA | 0.461538462 | 13 | 5.6768559 |
| GG | AA | AA | 0.212121212 | 33 | 14.4104803 |
| GG | AA | AC | 0 | 16 | 6.9868996 |
| GG | GG | AA | 0 | 2 | 0.8733624 |

The inventors next sought to demonstrate the use of a genomic selection model in prediction of the pathogen resistance trait and tested this on the training population of 234 diverse cannabis plants described above. The approach tested QTL1, where 22 markers were selected from the inventors' custom marker panel, described in the previous examples, at QTL1. The marker and the corresponding reference sequence are listed in Table 13.

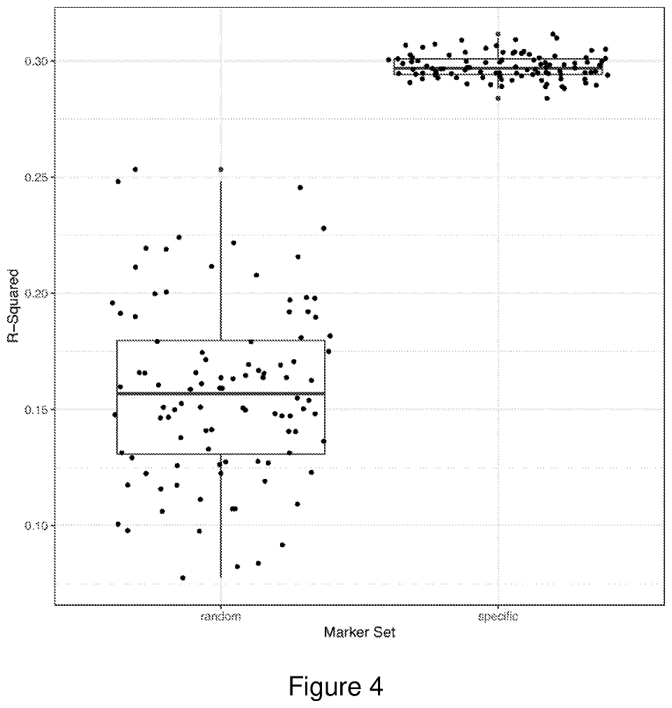

The model based on the 22 markers listed in Table 13 below tests if the markers together improve the prediction power for the selection of Botrytis resistant plants compared to 22 randomly selected markers. The inventors performed a multiple regression analysis with the allele as variable and Botrytis resistance as target using the random forest algorithm implemented in the ranger package (v 0.12.1, Wright and Zieger, 2017). The resulting R squares are derived from the comparison of the predictions from the developed model with the measured phenotype of the training population (FIG. 4). 100 permutations for the 22 specific markers and 100 permutations of the 22 random markers were conducted, each dot in FIG. 4 represents one of those permutations. The results of the genomic selection model demonstrate that the 22 specific markers in this region greatly improve the accuracy of selecting for Botrytis resistance for QTL1, R-squared of ˜0.3 over the use of random markers, R-squared of ˜0.15. Through the modeling approach, the inventors demonstrate that the selected markers for QTL1 significantly enhance the identification of plants resistant to botrytis. In fact, these markers explain twice the resistance variability compared to randomly selected markers, enabling a targeted selection of plants resistant to this disease.

TABLE 13
Detailed information of the 22 additional SNPs used in the genomic selection model for
selecting Botrytis resistance for QTL1 in Cannabis. The positions of the SNPs on
chromosome NC_044371.1 are provided with reference to the CS10 reference genome as
described herein The “context sequence” is provided with the SNP given in square
brackets. The first mentioned allele in square brackets is the reference allele based
on the CS10 genome and the identified alternative allele based on the polymorphism
detected is the second mentioned sequence in square brackets. All of the sequences and
alleles are provided with reference to the plus strand.

| QTL 1 Marker | | |
|---|---|---|
| Name | Position | Context Sequence [SNP] |
| MarkerSNP1 | 20668886 | AAGAACTTGATGATATTGCTAGCGCCAAAGACTCTATGAGCAATG |
| (common_710) | | GTGAA[C/T]TTAATAGGTTCGGTTGGCGGGAAATACGGCGCTAAAA |
| | | CGCACTTGTCGAC |
| | | (SEQ ID NO: 58) |
| MarkerSNP2 | 21138916 | CCCTATTTACTACAATGAGATGACAATGATCAAACACCATAAGTTG |
| | | GACG[T/C]ACCGGTGGCTGTCGAGGCCATCTCGAACACTGAGTAT |
| | | ATGGCCATGATGA |
| | | (SEQ ID NO: 59) |
| MarkerSNP3 | 21434575 | GAGACACTAACAGAGAATGGTCATGAGTGGATCGGTTTAGATATC |
| | | TCTGA[C/T]TCAATGCTTAGTATGTGGAAACTTATATTCTCGGTTAT |
| | | TTTGATTTGAAT |
| | | (SEQ ID NO: 60) |
| MarkerSNP4 | 21484705 | TATGCATCGTTTTACAAGAGCATCACCAATGATTGGGAGGACCAT |
| | | CTTGC[C/T]GTTAAGCACTTCTCCGTTGAAGGGCAGCTTGAATTCA |
| | | AGGCCATCCTTTT |
| | | (SEQ ID NO: 61) |
| MarkerSNP5 | 21514368 | TCTTTACAAACAGATATATGACACACATCTCTGCTCTCCTGAAAAT |
| | | CCAG[TGA/-]GATGATGACATTTTATCTTCATTACCATTTTTCTCACA |
| | | CCGAATAGCACC |
| | | (SEQ ID NO: 62) |
| MarkerSNP6 | 21549551 | ATATATACCTCCCTCTTCTCTGTACCAGCAGCATACTTCACCTTGA |
| | | AGAT[A/G]AAAATGTCTATGAGGGATAGACCTGTATTCCAGCAACA |
| | | TAAGTTGCCTCA |
| | | (SEQ ID NO: 63) |
| MarkerSNP7 | 21631563 | GAAGAGCGTAGTTTGGAGGAAACTCCAACCTGGGCTGTTGCTGT |
| | | AGTGGT[C/T]TTTGTGTTGATTATCATATCTATAATCATTGAACATG |
| | | TTATTCATCTCAT |
| | | (SEQ ID NO: 64) |
| MarkerSNP8 | 21632382 | GTCTTGGCTCTTTTCCATATTATATATTGCATTACCACCTTGGCTAT |
| | | TGG[C/T]AGAACCAAAGTGAGTAGTTTTTAAGTTTATCACTTTAGTT |
| | | TAACACATAT |
| | | (SEQ ID NO: 65) |
| MarkerSNP9 | 21762604 | AAATTCAAAGACATCATGACCTAAATAGTAATAAAACCCTACACCT |
| | | TGAT[A/G]GCGTACTTCCTCATGGGGAAGTAGACTTCCCTCTTCTT |
| | | TTCCCTTTCAGT |
| | | (SEQ ID NO: 66) |
| MarkerSNP10 | 22470941 | GTGGAACTAATCAGCAGTGAGTCAGGAGCCATGGTTGAAGTTAAA |
| (common_719) | | CACAT[A/T]GTGACACTTAAAAAGGATGAATTAAGTTCAGAAGTTGT |
| | | GATATCAAACTC |
| | | (SEQ ID NO: 67) |
| MarkerSNP11 | 22613893 | GTCCTACTCGACAACTCAAAAATCGAAAAACGTAACGTCCTTGAC |
| | | GGCAC[C/G]TTCATGCCCTCCACCTCCTTTACCGGCGGCCACCGC |
| | | GTGTGCGCTTATCT |
| | | (SEQ ID NO: 68) |
| MarkerSNP12 | 22681131 | CTTCAAGAAGCAATGAATGGTTGCCCAACATCAGTTACTGAAATC |
| | | ACTCC[A/G]GGGCTGCTTCTTAAAGATGCTGCTTATGCTGCTGCTG |
| | | CATATGTTTATTA |
| | | (SEQ ID NO: 69) |
| MarkerSNP13 | 22744351 | GGATTGACCAAAGATTTAGGAGGGTATTGGTCAATACAGTCTTTC |
| | | CACAC[G/A]TTGACATCATCCAAGTTCTTCATCACCTCCCACAAAC |
| | | AATTATTACACTT |
| | | (SEQ ID NO: 70) |
| MarkerSNP14 | 23036907 | CAGGAATGTCAAGACAATTTTGTGGCTTGTTCCAAGAGAAAATGG |
| | | CCATC[A/T]ATGAGGTGCACCTTGTTTAATTCCATGCACAAACTACT |
| | | AGTTTTATTAAG |
| | | (SEQ ID NO: 71) |
| MarkerSNP15 | 23123472 | TCTTCAGTCAGTATAACCTGTCCAAACAATAACAACTGAGGTGTCT |
| | | TTGT[T/C]TCCTCATTTGCCTTCAAACTTTGATTGGGATGCCCCATT |
| | | TTTAGCCAGCA |
| | | (SEQ ID NO: 72) |
| MarkerSNP16 | 23248673 | GGTGTCACTACCGGATTCGGTGCGACCTCCCACAGGAGAACCAA |
| | | AAATGG[C/T]GGAGCCCTTCAGAGGGAGCTAATTAGGTACGTACT |
| | | GTATCATTAATTAAC |
| | | (SEQ ID NO: 73) |
| MarkerSNP17 | 23326349 | CCTTTTAACCGCACATGCGAAGTAAATTTACCAGCTGGTGGTTTG |
| | | GATTC[A/G]GTAGACACCCTTCCTGCTATGTATGCAACGGTCCTCA |
| | | ATTTTTCTTCCTC |
| | | (SEQ ID NO: 74) |
| MarkerSNP18 | 23336680 | TGCCAAACTTGCTTATGAACAGATGGGGGTGGATTTTGAACTGCC |
| | | CCCTT[C/T]GCTTTTTTCACATTCTGACCTAATTCTTGCTGCCTAGT |
| | | GGTAAACACAGC |
| | | (SEQ ID NO: 75) |
| MarkerSNP19 | 23434116 | GGCTCTAAATGGAAGAAAAGATAGGGTTTTAGAGTATTTAAGATG |
| | | ACAAA[T/A]GCCGTACTTTCTGTATGACGTAATTTCCAAAGACATCA |
| | | GTCATCAATGTA |
| | | (SEQ ID NO: 76) |
| MarkerSNP20 | 23630372 | GCCGAGTTTCGTCTCCCACGAACACTCTCCAAATCAAAGAAGAAG |
| | | ATGCG[T/C]GTTCAGGCCTTGATCGATCAATTAGGTCTCAGAAATG |
| | | CAGCCAAGACAGT |
| | | (SEQ ID NO: 77) |
| MarkerSNP21 | 23661598 | AAATTTGGAGCTGTTTTAAAGAGCCTGAAGAACATAACATAGTTTC |
| (GBScompat_ | | CAGA[G/A]GTAACAGCAGCACGAACTGCAAGAGCATGCTTTACAG |
| common_137) | | CATTATCCCTTTT |
| | | (SEQ ID NO: 78) |
| MarkerSNP22 | 23715111 | GAGATGTTCCTACTCTCTTCAACTTTAAAATCTTTCATTAAAGCATC |
| | | ATT[C/A]TCAGTTTCATCATCACTTGAGATCACCATGCTAGATTCTT |
| | | CTTTGTACCA |
| | | (SEQ ID NO: 79) |

1. A method for characterizing a Cannabis spp. plant with respect to a pathogen resistance trait, optionally a Botrytis cinerea resistance trait, the method comprising the steps of:
(i) genotyping at least one plant with respect to at least one pathogen resistance QTL by detecting one or more polymorphisms associated with the pathogen resistance trait as defined in Table 2 or 9; and
(ii) characterizing the plant as having an increased pathogen resistance QTL, a decreased pathogen resistance QTL or an intermediate pathogen resistance QTL based on the genotype at the polymorphism.

2. The method of claim 1, wherein the polymorphism is selected from the group consisting of “common_3407”, “common_703”, “common_1691”, “common_701”, and combinations thereof as defined in Table 2 or 9.

3. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

4. The method of claim 3, wherein the molecular markers are for detecting polymorphisms at regular intervals within the at least one pathogen resistance QTL such that recombination can be excluded, or wherein the molecular markers are for detecting polymorphisms at regular intervals within the at least one pathogen resistance QTL such that recombination can be quantified to estimate linkage disequilibrium between a particular polymorphism and a pathogen resistance phenotype, optionally wherein the molecular markers are designed based on a context sequence for the polymorphism described in Table 3 or 10, or are selected from the primer pairs as defined in Table 4 or 11.

5. (canceled)

6. (canceled)

7. The method of claim 1, wherein the pathogen resistance QTL is selected from the group consisting of:
i. a quantitative trait locus having a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL;
ii. a quantitative trait locus having a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL;
iii. a quantitative trait locus defined by a single nucleotide polymorphism at position 79997 of NC_044373.1 of the CS10 genome and associated with pathogen resistance as defined in Table 2, or a genetic marker linked to the QTL; and
iv. a combination of any of (i) to (iii).

8. (canceled)

9. A method of producing a Cannabis spp. plant having a pathogen resistance trait of interest, optionally a Botrytis cinerea resistance trait, the method comprising the steps of:
(i) providing a donor parent plant having in its genome at least one pathogen resistance QTL characterized by one or more polymorphisms associated with the pathogen resistance trait of interest as defined Table 2 or 9;
(ii) crossing the donor parent plant having the at least one pathogen resistance QTL with at least one recipient parent plant to obtain a progeny population of cannabis plants;
(iii) screening the progeny population of cannabis plants for the presence of the at least one pathogen resistance QTL; and
(iv) selecting one or more progeny plants having the at least one pathogen resistance QTL, wherein the mature plant displays the pathogen resistance trait of interest.

10. The method of claim 9, further comprising:
(v) crossing the one or more progeny plants with the donor recipient plant; or
(vi) selfing the one or more progeny plants.

11. The method of claim 9, wherein the screening comprises genotyping at least one plant from the progeny population with respect to the at least one pathogen resistance QTL by detecting one or more polymorphisms associated with the pathogen resistance trait of interest as defined Table 2 or 9, and optionally wherein the method further comprises a step of genotyping the donor parent plant with respect to the at least one pathogen resistance QTL by detecting one or more polymorphisms associated with the pathogen resistance trait of interest as defined Table 2 or 9, prior to step (i).

12. (canceled)

13. The method of claim 11, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

14. The method of claim 13, wherein the molecular markers are for detecting polymorphisms at regular intervals within the at least one pathogen resistance QTL such that recombination can be excluded or such that recombination can be quantified to estimate linkage disequilibrium between a particular polymorphism and the pathogen resistance trait of interest, optionally wherein the molecular markers are designed based on a context sequence for the polymorphism in Tables 3 or 10, or are selected from the primer pairs as defined in Table 4 or 11.

15. (canceled)

16. The method of claim 9, wherein the polymorphism is selected from the group consisting of “common_3407”, “common_703”, “common_1691”, “common_701”, and combinations thereof as defined in Table 2 or 9.

17. The method of claim 9, wherein the pathogen resistance QTL is an increased pathogen resistance QTL, a decreased pathogen resistance QTL, or an intermediate pathogen resistance QTL as defined in Table 2 or 9.

18. The method of claim 9, wherein the pathogen resistance QTL is selected from the group consisting of:
i. a quantitative trait locus having a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL;
ii. a quantitative trait locus having a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL;
iii. a quantitative trait locus defined by a single nucleotide polymorphism at position 79997 of NC_044373.1 of the CS10 genome and associated with pathogen resistance as defined in Table 2, or a genetic marker linked to the QTL; and
iv. a combination of any of (i) to (iii).

19. (canceled)

20. A method of producing a Cannabis spp. plant that has a pathogen resistance trait of interest, optionally a Botrytis cinerea resistance trait, the method comprising introducing at least one pathogen resistance QTL characterized by one or more polymorphisms associated with the pathogen resistance trait of interest as defined in Table 2 or 9 into a Cannabis spp. plant, wherein said QTL is associated with the pathogen resistance trait of interest in the plant.

21. The method of claim 20, wherein introducing the at least one pathogen resistance QTL comprises crossing a donor parent plant having the at least one pathogen resistance QTL with a recipient parent plant, or genetically modifying the Cannabis spp. plant.

22. (canceled)

23. The method of claim 20, wherein the pathogen resistance QTL is selected from the group consisting of:
i. a quantitative trait locus having a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL;
ii. a quantitative trait locus having a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL;
iii. a quantitative trait locus defined by a single nucleotide polymorphism at position 79997 of NC_044373.1 of the CS10 genome and associated with pathogen resistance as defined in Table 2, or a genetic marker linked to the QTL; and
iv. a combination of any of (i) to (iii).

24-27. (canceled)

28. A Cannabis spp. plant comprising at least one pathogen resistance QTL characterized by one or more polymorphisms associated with a pathogen resistance trait of interest as defined in Table 2 or 9, wherein said pathogen resistance QTL is associated with the pathogen resistance trait of interest in the plant, optionally wherein the pathogen resistance trait of interest is a Botrytis cinerea resistance trait.

29-33. (canceled)

34. The Cannabis spp. plant of claim 28, wherein the pathogen resistance QTL is selected from the group consisting of:
i. a quantitative trait locus that controls a pathogen resistance trait in Cannabis spp., wherein the quantitative trait locus has a sequence that corresponds to nucleotides 18901130 to 23661598 of NC_044371.1 of the CS10 genome and is defined by one or more polymorphisms associated with pathogen resistance as defined in Table 2 or 9, or a genetic marker linked to the QTL;
ii. a quantitative trait locus that controls a pathogen resistance trait in Cannabis spp., wherein the quantitative trait locus has a sequence that corresponds to nucleotides 86855033 to 90075725 of NC_044375.1 of the CS10 genome and is defined by one or more polymorphisms associated with pathogen resistance as defined in Table 2, or a genetic marker linked to the QTL; and
iii. a quantitative trait locus that controls a pathogen resistance trait in Cannabis spp., wherein the quantitative trait locus is defined by a single nucleotide polymorphism at position 79997 of NC_044373.1 of the CS10 genome and is associated with pathogen resistance as defined in Table 2, or a genetic marker linked to the QTL.


SEQUENCE LISTING (79 sequences)

SEQ ID NO: 1 - DNA, 23 bp, synthetic construct
  source 1..23: mol_type = other DNA
attgacatcgtcgctgtcaacga

SEQ ID NO: 2 - DNA, 21 bp, synthetic construct
  source 1..21: mol_type = other DNA
accccactcgttgtcgtacca

SEQ ID NO: 3 - DNA, 21 bp, synthetic construct
  source 1..21: mol_type = other DNA
caattgagatttgcccacaag

SEQ ID NO: 4 - DNA, 24 bp, synthetic construct
  source 1..24: mol_type = other DNA
gatggatccagtggtaccgagcat

SEQ ID NO: 5 - DNA, 403 bp, Cannabis sativa
  source 1..403: mol_type = genomic DNA
  variation 202: allele = N is C or A
gaaggtctaaaatcttgaaaatgctgcgacttatataaaggaaaataaaatgaatgtttaaatttagtttcaatttcaatttcaatttatagtcttaagcaggaagcagggcccttcatcttccagaaattcaatggagaagtagtgagactcagtgtgagagcgagagatggaagccgttgcagaggggctttggggactngcggactaccacgagaagaaaggagacgtagggaaagctgtgaagtgtctcgaagccatttgccagagtcaggtcaccttcttccccatcgtcgaggtcaagactcgtcttcgaatcgctgctttacttctcaagcattctcataatgttaatcacgctaagtctcacctcgaacgatctcagttgcttctcaagaacatc

SEQ ID NO: 6 - DNA, 403 bp, Cannabis sativa
  source 1..403: mol_type = genomic DNA
  variation 202: allele = N is G or A
atttatctgcaaaagatataatgcacaaaaacaaacctgttcagcaatgctagctgcttcagcatatgcatcttgaatattctcagcaagcctgtggagaaatatgtttgatattcctccggccgcaacaataaatggaccagtggccaatgttatgagtgcaatctgccaacagttgacaaatccaataacaagaccagcnaaaaatgtagccatgttgtgaacataatttccaacctgtaatcaaagaggaacacagactttagacaagaaatttgaatcaacaaatcatatagaaagctgaaatttttcataacaaataaaaattcccaaaagggaacatgagctagctaatataaattagcataaaattaccttttcacttaaagcggactgaatgagc

SEQ ID NO: 7 - DNA, 403 bp, Cannabis sativa
  source 1..403: mol_type = genomic DNA
  variation 202: allele = N is A or T
tcctttctttttggagattcaactctaatgacattttaagtagagttttcttcttggttaatggtactaggattaaattattgctatgtacaatggtagaactagaagccaagttactaacacaaaagcttagttcttttgagaagtgcaggtggaactaatcagcagtgagtcaggagccatggttgaagttaaacacatngtgacacttaaaaaggatgaattaagttcagaagttgtgatatcaaactctagctctgctccagttcaattgactgggtgtgttctgagccatctgagggtgagctctcctgatgcaacttatgcaatcggattagaaggatcggatttcttctccaggcctccattttcatcaaactttggtattgttcctccagacttt

SEQ ID NO: 8 - DNA, 403 bp, Cannabis sativa
  source 1..403: mol_type = genomic DNA
  variation 202: allele = N is G or T
agaaataaagatggcggtgattggttgaaaggaaaaacagtagcagaagcaagtgcctgcagtgagaaagtgggaaattttacctgggaaagtgtagctgatcggcgagagaatagtatacaaatgccattcatactggatgataatattgcagcgaacagaaacaaagataacagtgattggttgaaaggaaaagcagtancagaaacaagtgccttcagtgagaaagttggaaatttttcttgggaaaatgtagctgatcgaccagagaaaagtattcaagtatttttcaatggaaagattgaaaaatttccctggaaaaatgtagttggccaacgggaaaatactagtcaaatcccatttattaacgatgcagccaatggagagaatgatgacagttctt

SEQ ID NO: 9 - DNA, 403 bp, Cannabis sativa
  source 1..403: mol_type = genomic DNA
  variation 202: allele = n is G or A
tgcagctcactgccttaaaccgcatcttttcaacatatagatctgcagaaattttaatactgtgttatataatatgatcattcttttaaaaaaaaaatcattaaaatggagatagcaacttggctaaaactcacccattacacaagtgttcaaatttggagctgttttaaagagcctgaagaacataacatagtttccagangtaacagcagcacgaactgcaagagcatgctttacagcattatccctttttgcttcccttgataatctgaactcaatttcacaacaatattgaaggtcattacaagagtttcaacatagtagaaggatggtaaatagctaatgaaatttcctcaaatcataactgaccttgacatggatgatacaagatctctgttgttac

SEQ ID NO: 10 - DNA, 403 bp, Cannabis sativa
  source 1..403: mol_type = genomic DNA
  variation 202: allele = N is G or C
gctacattgttttggtgtacctaatggataacattagttgggaattagggtttgggatttgtcttgccatgacagcacttggactaatttcgtttttgcttggaaagaatttctatcgattcgttaagcctaagggaagcccatttgtgagcatggctagagttgtcgttgcagctattcggaatagaaaattgtcactaantgatcgtgctttaacaaatacatatacatcgggtatcattgttcacacaaaatcaccaagtaaacgtttcaggtatattatatataaaatatccaactaaattatatattgtagaaataaataatagtaatgattaactcccttaaaatttcaaagtagtattaagggaaaaagaaggtcctcaaatttactgttgaattg

SEQ ID NO: 11 - DNA, 403 bp, Cannabis sativa
  source 1..403: mol_type = genomic DNA
  variation 202: allele = N is G or A
ctagatttctttgtcttgaaatttatttttctttttgatctgtttcttctggttctgatctgtaaggatccgtgtatttcactcttgggtttttttttttttttatcagtttaggggatttgagttgcgatttttgtgtcgtttgactgttttggtttgcgatctggtgctgatttgtgtcgtttgtgcgtttttgtagctncgtctgtgtaataatggcgtcgaaacggatcttgaaggagctcaaggatttgcagaaagatcctcctacctcttgcagcgccggtatatttcgttaccttagtattgttctcttttatatgcgtagcttatattgttaatccggtgaacggaaagctttatctttctgtgtgtttatataatatattatctatttatttat

SEQ ID NO: 12 - DNA, 403 bp, Cannabis sativa
  source 1..403: mol_type = genomic DNA
  variation 202: allele = N is C or T
ccaaaatgagcacatgaaaaaataaagtagaattaaggaaaatgttctatacccaaattaatacatagtcgaatggttaattggactatattataaaatttaattatttatttataggattaattgtttgaataaatatatatatacctgcaagaacttgatgatattgctagcgccaaagactctatgagcaatggtgaanttaataggttcggttggcgggaaatacggcgctaaaacgcacttgtcgacacatcggcggcgaaggattttgcaggcagcgcaggggctcatgacgatagctggtggtggaggcggtggcggaggaggagttggactaacattattattactagtactggtgggagaagattgagaggagggtggagaagataactgagat

SEQ ID NO: 13 - DNA, 403 bp, Cannabis sativa
  source 1..403: mol_type = genomic DNA
  variation 202: allele = N is G or A
ttgtagaggatctgtgaaggggctttctgttttacttgagcctgtatttttgtcacaacgggatgtgcaagaactcttgtggatcagctttctcgccagctctatccattacccttctcatcattgatcttattgaatctggttgatgaaaatggtccagatgaataataaagctcccgtaaccaatgatgagctaactganctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacat

SEQ ID NO: 14 - DNA, 403 bp, Cannabis sativa
  source 1..403: mol_type = genomic DNA
  variation 202: allele = N is A or G
taatatattgaaattgattttgtgcatttaacaattttcatgtcattttgtgcacccctctcctataatttttaattattatttatatgttttgttgattaggaaatatgaagtttaattttatgtctatgtttcagatacaatggcggttcccctactcaccaagaagattgtgaagaagcgcgtcaagcagttcaagaanccccaatgggacaggaagatatccgtcaaggtttgtagttcaccaattattcctcatttgtaggttttattatgacttaatcagtatgcaagtatttgtaggtacaagtgtttatcttctcatctattttgtaaagacaatgttgatagctttgtttattttaacattttcctgttctggttttcccttttgattttagtt

SEQ ID NO: 15 - DNA, 403 bp, Cannabis sativa
  source 1..403: mol_type = genomic DNA
  variation 202: allele = N is T or C
aacaatgaggagttgttgaataacgaattctacattggacttaggcaaaggagagcaactgggaaggtctagtaacttcttgttacattttattgatgtgtaaggttatagtttccactttcaaaatgcagttaaatcagccttgagaatctgtttgcattatgtacaggaatattctgagcttttgcacgagttcatgacngcagttaagcaaaactatggggaaaaagttcttgtgcaggtaaacatttgattttaaagctacattttcagcatatgcatggatttacttgaggttttcagcattctatttctgttgatgtttgcagttcgaagattttgcaaaccacaatgcttttgatctgctagcgaaatatggaaccacgcatcttgtcttcaacga

SEQ ID NO: 16 - DNA, 20 bp, synthetic construct
  source 1..20: mol_type = other DNA
gaagcagggcccttcatctt

SEQ ID NO: 17 - DNA, 21 bp, synthetic construct
  source 1..21: mol_type = other DNA
agcaactgagatcgttcgagg

SEQ ID NO: 18 - DNA, 20 bp, synthetic construct
  source 1..20: mol_type = other DNA
aagcagggcccttcatcttc

SEQ ID NO: 19 - DNA, 20 bp, synthetic construct
  source 1..20: mol_type = other DNA
tttgatattcctccggccgc

SEQ ID NO: 20 - DNA, 22 bp, synthetic construct
  source 1..22: mol_type = other DNA
gctcattcagtccgctttaagt

SEQ ID NO: 21 - DNA, 20 bp, synthetic construct
  source 1..20: mol_type = other DNA
ttctcagcaagcctgtggag

SEQ ID NO: 22 - DNA, 22 bp, synthetic construct
  source 1..22: mol_type = other DNA
gctagctcatgttcccttttgg

SEQ ID NO: 23 - DNA, 20 bp, synthetic construct
  source 1..20: mol_type = other DNA
tgagaagtgcaggtggaact

SEQ ID NO: 24 - DNA, 20 bp, synthetic construct
  source 1..20: mol_type = other DNA
aggcctggagaagaaatccg

SEQ ID NO: 25 - DNA, 20 bp, synthetic construct
  source 1..20: mol_type = other DNA
ggcctggagaagaaatccga

SEQ ID NO: 26 - DNA, 20 bp, synthetic construct
  source 1..20: mol_type = other DNA
aagtgtagctgatcggcgag

SEQ ID NO: 27 - DNA, 20 bp, synthetic construct
  source 1..20: mol_type = other DNA
tctccattggctgcatcgtt

SEQ ID NO: 28 - DNA, 20 bp, synthetic construct
  source 1..20: mol_type = other DNA
gtgtagctgatcggcgagag

SEQ ID NO: 29 - DNA, 20 bp, synthetic construct
  source 1..20: mol_type = other DNA
cagctcactgccttaaaccg

SEQ ID NO: 30 - DNA, 21 bp, synthetic construct
  source 1..21: mol_type = other DNA
tgctgtaaagcatgctcttgc

SEQ ID NO: 31 - DNA, 20 bp, synthetic construct
  source 1..20: mol_type = other DNA
tcgtgctgctgttacctctg

SEQ ID NO: 32 - DNA, 21 bp, synthetic construct
  source 1..21: mol_type = other DNA
tcgttaagcctaagggaagcc

SEQ ID NO: 33 - DNA, 22 bp, synthetic construct
  source 1..22: mol_type = other DNA
tttgaggaccttctttttccct

SEQ ID NO: 34 - DNA, 20 bp, synthetic construct
  source 1..20: mol_type = other DNA
gggaagcccatttgtgagca

SEQ ID NO: 35 - DNA, 22 bp, synthetic construct
  source 1..22: mol_type = other DNA
tccgtgtatttcactcttgggt

SEQ ID NO: 36 - DNA, 23 bp, synthetic construct
  source 1..23: mol_type = other DNA
aagataaagctttccgttcaccg

SEQ ID NO: 37 - DNA, 21 bp, synthetic construct
  source 1..21: mol_type = other DNA
gataaagctttccgttcaccg

SEQ ID NO: 38 - DNA, 20 bp, synthetic construct
  source 1..20: mol_type = other DNA
ttgctagcgccaaagactct

SEQ ID NO: 39 - DNA, 20 bp, synthetic construct
  source 1..20: mol_type = other DNA
tcttctccaccctcctctca

SEQ ID NO: 40 - DNA, 21 bp, synthetic construct
  source 1..21: mol_type = other DNA
tcttctccaccctcctctcaa

SEQ ID NO: 41 - DNA, 20 bp, synthetic construct
  source 1..20: mol_type = other DNA
tggatcagctttctcgccag

SEQ ID NO: 42 - DNA, 20 bp, synthetic construct
  source 1..20: mol_type = other DNA
gctggccagaatgactcctc

SEQ ID NO: 43 - DNA, 20 bp, synthetic construct
  source 1..20: mol_type = other DNA
tgtggatcagctttctcgcc

SEQ ID NO: 44 - DNA, 20 bp, synthetic construct
  source 1..20: mol_type = other DNA
agatacaatggcggttcccc

SEQ ID NO: 45 - DNA, 23 bp, synthetic construct
  source 1..23: mol_type = other DNA
tcaaaagggaaaaccagaacagg

SEQ ID NO: 46 - DNA, 20 bp, synthetic construct
  source 1..20: mol_type = other DNA
gatacaatggcggttcccct

SEQ ID NO: 47 - DNA, 20 bp, synthetic construct
  source 1..20: mol_type = other DNA
ggagagcaactgggaaggtc

SEQ ID NO: 48 - DNA, 23 bp, synthetic construct
  source 1..23: mol_type = other DNA
ggtttgcaaaatcttcgaactgc

SEQ ID NO: 49 - DNA, 20 bp, synthetic construct
  source 1..20: mol_type = other DNA
aggcaaaggagagcaactgg

SEQ ID NO: 50 - DNA, 22 bp, synthetic construct
  source 1..22: mol_type = other DNA
tcgaactgcaaacatcaacaga

SEQ ID NO: 51 - DNA, 111 bp, Cannabis sp.
  source 1..111: mol_type = other DNA
tgtatcttgggttcgactacatgggtactcttcttagacttgcacatgagtacacacaaatacacacaaattgattaagcacaaattttgatctttgtgatggtgattgtt

SEQ ID NO: 52 - DNA, 113 bp, Cannabis sp.
  source 1..113: mol_type = other DNA
tgtatcttgggttcgactacatgggtactcttcttagacttgcacatgagtacacacacaaatacacacaaattgattaagcacaaattttgatctttgtgatggtgattgtt

SEQ ID NO: 53 - DNA, 101 bp, Cannabis sp.
  source 1..101: mol_type = other DNA
  variation 51: allele = n = c/a
aatcacttattactaagcaagaaagagtctcaaagagaccgattggatcancggaatcggattccgttcgtcggaaaaagttcagaggcgttcgccgaaga

SEQ ID NO: 54 - DNA, 403 bp, Cannabis sp.
  source 1..403: mol_type = other DNA
  variation 202: allele = n = c/t
tataaaaactctcctccatatgatcggctcctataccattactataactaactaactaactagctatttatatatatatatatatatatattataatataaaaatattatatgtatgtgaatgtgatgaacataatataaatcatatatatgtaaccaaagaaactgatttcccaagctgaagaatcttgatatcattttcnaaaggtgtgaggttcaaatccaaacacaaaaccctattgttcttcttattcaccatcgcggccggccgccgtacttgcaaagaaccggcaatatcaacaggcggtggccgtgtggcggccgaggccgctgacaaagcatgatgatgagtctggtcggtcaaggcggctctatggcgcctcatgtggccccccaaggcttgg

SEQ ID NO: 55 - DNA, 21 bp, synthetic construct
  source 1..21: mol_type = other DNA
tcctccatatgatcggctcct

SEQ ID NO: 56 - DNA, 20 bp, synthetic construct
  source 1..20: mol_type = other DNA
ccaccgcctgttgatattgc

SEQ ID NO: 57 - DNA, 20 bp, synthetic construct
  source 1..20: mol_type = other DNA
gccaccgcctgttgatattg

SEQ ID NO: 58 - DNA, 101 bp, Cannabis sp.
  source 1..101: mol_type = other DNA
  variation 51: allele = n = c/t
aagaacttgatgatattgctagcgccaaagactctatgagcaatggtgaanttaataggttcggttggcgggaaatacggcgctaaaacgcacttgtcgac

SEQ ID NO: 59 - DNA, 101 bp, Cannabis sp.
  source 1..101: mol_type = other DNA
  variation 51: allele = n = t/c
ccctatttactacaatgagatgacaatgatcaaacaccataagttggacgnaccggtggctgtcgaggccatctcgaacactgagtatatggccatgatga

SEQ ID NO: 60 - DNA, 101 bp, Cannabis sp.
  source 1..101: mol_type = other DNA
  variation 51: allele = n = c/t
gagacactaacagagaatggtcatgagtggatcggtttagatatctctgantcaatgcttagtatgtggaaacttatattctcggttattttgatttgaat

SEQ ID NO: 61 - DNA, 101 bp, Cannabis sp.
  source 1..101: mol_type = other DNA
  variation 51: allele = n = c/t
tatgcatcgttttacaagagcatcaccaatgattgggaggaccatcttgcngttaagcacttctccgttgaagggcagcttgaattcaaggccatcctttt

SEQ ID NO: 62 - DNA, 103 bp, Cannabis sp.
  source 1..103: mol_type = other DNA
  variation 51..53: note = Allele = deletion of residues 51 to 53
tctttacaaacagatatatgacacacatctctgctctcctgaaaatccagtgagatgatgacattttatcttcattaccatttttctcacaccgaatagcacc

SEQ ID NO: 63 - DNA, 101 bp, Cannabis sp.
  source 1..101: mol_type = other DNA
  variation 51: allele = n = a/g
atatatacctccctcttctctgtaccagcagcatacttcaccttgaagatnaaaatgtctatgagggatagacctgtattccagcaacataagttgcctca

SEQ ID NO: 64 - DNA, 101 bp, Cannabis sp.
  source 1..101: mol_type = other DNA
  variation 51: allele = n = c/t
gaagagcgtagtttggaggaaactccaacctgggctgttgctgtagtggtntttgtgttgattatcatatctataatcattgaacatgttattcatctcat

SEQ ID NO: 65 - DNA, 101 bp, Cannabis sp.
  source 1..101: mol_type = other DNA
  variation 51: allele = n = c/t
gtcttggctcttttccatattatatattgcattaccaccttggctattggnagaaccaaagtgagtagtttttaagtttatcactttagtttaacacatat

SEQ ID NO: 66 - DNA, 101 bp, Cannabis sp.
  source 1..101: mol_type = other DNA
  variation 51: allele = n = a/g
aaattcaaagacatcatgacctaaatagtaataaaaccctacaccttgatngcgtacttcctcatggggaagtagacttccctcttcttttccctttcagt

SEQ ID NO: 67 - DNA, 101 bp, Cannabis sp.
  source 1..101: mol_type = other DNA
  variation 51: allele = n = a/t
gtggaactaatcagcagtgagtcaggagccatggttgaagttaaacacatngtgacacttaaaaaggatgaattaagttcagaagttgtgatatcaaactc

SEQ ID NO: 68 - DNA, 101 bp, Cannabis sp.
  source 1..101: mol_type = other DNA
  variation 51: allele = n = c/g
gtcctactcgacaactcaaaaatcgaaaaacgtaacgtccttgacggcacnttcatgccctccacctcctttaccggcggccaccgcgtgtgcgcttatct

SEQ ID NO: 69 - DNA, 101 bp, Cannabis sp.
  source 1..101: mol_type = other DNA
  variation 51: allele = n = a/g
cttcaagaagcaatgaatggttgcccaacatcagttactgaaatcactccngggctgcttcttaaagatgctgcttatgctgctgctgcatatgtttatta

SEQ ID NO: 70 - DNA, 101 bp, Cannabis sp.
  source 1..101: mol_type = other DNA
  variation 51: allele = n = g/a
ggattgaccaaagatttaggagggtattggtcaatacagtctttccacacnttgacatcatccaagttcttcatcacctcccacaaacaattattacactt

SEQ ID NO: 71 - DNA, 101 bp, Cannabis sp.
  source 1..101: mol_type = other DNA
  variation 51: allele = n = a/t
caggaatgtcaagacaattttgtggcttgttccaagagaaaatggccatcnatgaggtgcaccttgtttaattccatgcacaaactactagttttattaag

SEQ ID NO: 72 - DNA, 101 bp, Cannabis sp.
  source 1..101: mol_type = other DNA
  variation 51: allele = n = t/c
tcttcagtcagtataacctgtccaaacaataacaactgaggtgtctttgtntcctcatttgccttcaaactttgattgggatgccccatttttagccagca

SEQ ID NO: 73 - DNA, 101 bp, Cannabis sp.
  source 1..101: mol_type = other DNA
  variation 51: allele = n = c/t
ggtgtcactaccggattcggtgcgacctcccacaggagaaccaaaaatggnggagcccttcagagggagctaattaggtacgtactgtatcattaattaac

SEQ ID NO: 74 - DNA, 101 bp, Cannabis sp.
  source 1..101: mol_type = other DNA
  variation 51: allele = n = a/g
ccttttaaccgcacatgcgaagtaaatttaccagctggtggtttggattcngtagacacccttcctgctatgtatgcaacggtcctcaatttttcttcctc

SEQ ID NO: 75 - DNA, 101 bp, Cannabis sp.
  source 1..101: mol_type = other DNA
  variation 51: allele = n = c/t
tgccaaacttgcttatgaacagatgggggtggattttgaactgcccccttngcttttttcacattctgacctaattcttgctgcctagtggtaaacacagc

SEQ ID NO: 76 - DNA, 101 bp, Cannabis sp.
  source 1..101: mol_type = other DNA
  variation 51: allele = n = t/a
ggctctaaatggaagaaaagatagggttttagagtatttaagatgacaaangccgtactttctgtatgacgtaatttccaaagacatcagtcatcaatgta

SEQ ID NO: 77 - DNA, 101 bp, Cannabis sp.
  source 1..101: mol_type = other DNA
  variation 51: allele = n = t/c
gccgagtttcgtctcccacgaacactctccaaatcaaagaagaagatgcgngttcaggccttgatcgatcaattaggtctcagaaatgcagccaagacagt

SEQ ID NO: 78 - DNA, 101 bp, Cannabis sp.
  source 1..101: mol_type = other DNA
  variation 51: allele = n = g/a
aaatttggagctgttttaaagagcctgaagaacataacatagtttccagangtaacagcagcacgaactgcaagagcatgctttacagcattatccctttt

SEQ ID NO: 79 - DNA, 101 bp, Cannabis sp.
  source 1..101: mol_type = other DNA
  variation 51: allele = n = c/a
gagatgttcctactctcttcaactttaaaatctttcattaaagcatcattntcagtttcatcatcacttgagatcaccatgctagattcttctttgtacca